(19) United States
(12) Patent Application Publication
(10) Pub. No.: US 2004/0068181 A1
(43) Pub. Date: Apr. 8, 2004

(54) Image forming apparatus, image forming method and image forming system

(75) Inventor: Toshihisa Takeyama, Tokyo (JP)

Correspondence Address: MUSERLIAN AND LUCAS AND MERCANTI, LLP, 475 PARK AVENUE SOUTH, NEW YORK, NY, 10016, US

(73) Assignee: Konica Corporation, Tokyo (JP)

(21) Appl. No.: 10/663,522

(22) Filed: Sep. 15, 2003

(30) Foreign Application Priority Data
Sep. 24, 2002 (JP) JP2002-277119

(51) Int. Cl. A61B 5/05

(52) U.S. Cl. 600/425

(57) ABSTRACT

An image forming apparatus for forming an image based on digitalized medical image data, comprising: a first image forming material-supplying section supplying a first image forming material; a second image forming material-supplying section supplying a second image forming material, which is different from the first image forming material; a selecting section selecting an image forming material to be output from the first and second image forming materials; a converting section converting the digitalized medical image data to an outputting image data, which is suited to the selected image forming material; an outputting section outputting the outputting image data onto the selected image forming material; and a post-processing section conducting a post-processing to the selected image forming material to form a final image.

### BACKGROUND OF THE INVENTION

The present invention relates to an image forming apparatus, an image forming method, and an image forming system.

In recent years, due to the advance in medical technology, it has become possible to treat various diseases. The aforesaid advance in medical technology is pronounced especially in the progress of various types of diagnostic apparatuses such as magnetic resonance imaging (MRI), computed tomography (CT), multi-slice CT apparatus, positron emission computed tomography (PET), nuclear medicine diagnosis, ultrasonic image diagnosis, an angiographic X-ray diagnostic apparatus, computed tomography (employing an X-ray CT apparatus), RI diagnosis (employing a scintillation camera), mammography, electron endoscope diagnosis, fundus camera diagnosis, and radiation image reading diagnosis (employing a CR apparatus and an FPD apparatus), which are employed for physiological tests in clinical tests. Further, blood tests and pathologic tests of specimens are performed employing a microscope fitted with CCD, whereby it is possible to determine in a short time the shape, size and number by processing the resulting images. As a result, it has become possible to achieve early diagnosis as well as early treatment.

On the other hand, in the aforesaid image diagnostic apparatuses, image information is often provided in the form of digital signals. When diagnosis is conducted employing these medical images, some diagnostic images are viewed on a CRT or a liquid crystal monitor, or a diagnostic process is observed by displaying the difference before and after treatment. From the viewpoint of reliability, most image information is outputted onto image forming materials in the form of a hard copy for viewing. Medical image forming apparatuses which output such medical image information onto image forming materials utilize a silver salt color forming system in which images are formed under laser scanning exposure, a thermosensitive color forming system employing thermal printers, a thermosensitive fusion transfer system, or a thermosensitive sublimation thermal transfer system, and an ink jet recording system employing ink jet printers. These are appropriately selected and used corresponding to the required medical images.

When medical image data in digital form are outputted, employing the silver salt color forming system while employing any of the various diagnostic apparatuses previously described, the optimal maximum density of images which are employed for diagnosis occasionally varies depending on various diagnostic apparatuses.

In order to overcome such drawbacks, for example, proposed is an image forming material which increases the maximum density. As examples of such image forming materials, the image forming material described in Japanese Patent Application Open to Public Inspection (hereinafter referred to as JP-A) No. 2000-10231 (Paragraph No. 0140-0173), JP-A No. 2000-10232 (Paragraph No. 0152-0186) and JP-A No. 2000-10231 (paragraph Nos. 0140-0143) can be cited. Further, there is a need to control the tone of images used for diagnosis, depending on the personal preference of the doctor who attempts diagnosis via these images or on the type of light source and the diffusing plate of the viewing box. In order to meet such needs, proposed are image forming materials of which image tone is variously controlled. For example, image forming materials described in JP-A No. 10-268465 (paragraph Nos. 0011-0021), JP-A No. 11-231460 (Paragraph Nos. 0078-0125), JP-A No. 11-288057 (Paragraph Nos. 0019-0050), JP-A No. 2001-330923 (Paragraph Nos. 0016-0074), JP-A No. 2002-169249 (Paragraph Nos. 0117-0174), European Patent Nos. 1,004,929 (page 18, line 53-page 19, line 24) and U.S. Pat. No. 6,174,657 (column 26, line 19 -column 33, line 53) are cited.

However, an image forming apparatus employing the aforesaid image forming materials commonly employs one size of the material even though there are different sizes available. Accordingly, when an image forming material is changed, it has required each of the image forming apparatuses to be subjected to new tone correction and the like.

### SUMMARY OF THE INVENTION

From the viewpoint of the aforesaid problems, the present invention was achieved. An aspect of the present invention is to provide an image forming apparatus, an image forming method, and an image forming system which are suitable for the simultaneous use of plural image forming materials which is different from each other, and in more detail to provide an image forming apparatus, an image forming method and an image forming system which are capable of outputting images onto various types of image forming materials while using the same apparatus.

The present invention was achieved employing the structures below.

A first structure of the invention is an image forming apparatus for forming an image on an image forming material based on digitalized medical image data. The image forming apparatus comprises a first image forming material-supplying section supplying a first image forming material;

a second image forming material-supplying section supplying a second image forming material, which is different from the first image forming material;

a selecting section selecting an image forming material to be output from the first and second image forming materials;

a converting section converting the digitalized medical image data to an outputting image data, which is suited to the selected image forming material;

an outputting section outputting the outputting image data onto the selected image forming material; and

a post-processing section conducting a post-processing to the selected image forming material to form a final image.

In the image forming apparatus, it is preferable that each of the first image forming material and the second image forming material is a tray.

In the image forming apparatus, it is preferable that the first image forming material and the second image forming material are different in a color tone or a maximum density from each other when an image is formed on each of the first image forming materials and the second image forming material in same condition. Further, it is also preferable that the first image forming material and the second image forming material are different in at least one of sensitivity, transmittance and a gradient from each other. Still further, it is also preferable that one of the first image forming material and the second image forming material has a reflective support and the other has a transparent support.

In the image forming apparatus, the outputting section is a photo-writing device utilizing a laser scan exposure. Further, it is more preferable that a system of the laser scan exposure of the photo-writing device is a laser scan exposing system in which an angle of an exposed surface and a laser beam is not substantially perpendicular, a longitudinally multi laser scan exposing system utilizing a laser beam has plural exposing wavelengths, or a laser scan exposing system in which the laser scan exposure is conducted by two or more of laser beams. Still further, it is also preferable that an emission wavelength of a laser source utilized in the photo-writing device is in a range of 600 to 1200 nm, and is more preferably 750 to 850 nm.

In the image forming apparatus, it is preferable that the post-processing section is a heat-processing device. Further, it is more preferable that the image forming apparatus suffices the following relationship,

1200≦t×T≦2600 [sec·° C.]

wherein t is a time period, the selected image forming material being subjected to a heat-processing; and T is a temperature of a surface of the heat-processing device where the selected image forming material contacts. Still further, it is more preferable that the image forming apparatus suffices the following relationship,

1480≦t×T≦1860 [sec·° C.]

In the image forming apparatus, it is preferable that the converting section has at least one of a resolution-converting function, a gradient-converting function, a color-converting function and an LUT (Look-Up Table)-converting function. Further, it is more preferable that the converting section has at least one of the resolution converting function, the gradient-converting function and the color-converting function. It is also preferable that the converting section has the LUT-converting function.

A second structure of the invention is an image forming method for forming an image on an image forming material based on a digitalized medical image data. The image forming method comprises:

selecting an image forming material to be output from plural image forming materials, which are different from each other;

converting the digitalized medical image data to an outputting image data, which is suited to the selected image forming material;

outputting the outputting image data onto the selected image forming material; and

conducting a post-processing to the selected image forming material to form a final image.

In the image forming method, it is preferable that the plural image forming materials are different in a color tone or a maximum density from each other when an image is formed on each of the plural image forming materials in same condition. Further, In the image forming method, it is also preferable that the plural image forming materials are different in sensitivity, transmittance or gradient from each other. Still further, it is preferable that the plural image forming materials include an image forming material having a reflective support and an image forming material having a transparent support.

In the image forming method, the outputting step is conducted by a laser scan exposure.

In the image forming method, it is preferable that the post-processing is a heat processing. Further, it is more preferable that the image forming method suffices the following relationship,

1200≦t×T≦2600 [sec·° C.]

wherein t is a time period, the selected image forming material being subjected to a heat-processing; and T is a temperature of a surface of a heat-processing device where the selected image forming material contacts. Still further, it is still more preferable that the image forming method suffices the following relationship,

1480≦t×T≦1860 [sec·° C.].

In the image forming method, it is preferable that the converting step is directly determined in accordance with a result of the selecting step.

In the image forming method, it is preferable that the converting step includes at least one of the steps of converting resolution of the digitalized medical image data, converting gradient of the digitalized medical image data, converting color of the digitalized medical image data, and converting LUT of the digitalized medical image data. Further, it is more preferable that the converting step includes at least one of the step of converting resolution of the digitalized medical image data, converting gradient of the digitalized medical image data and converting color of the digitalized medical image data. It is also preferable that the converting step includes the step of converting LUT of the digitalized medical image data.

In the image forming method, it is preferable that the image forming method further comprises displaying the outputting image data on a displaying section. Further, it is more preferable that the image forming method further comprises correcting the outputting image data for representing the outputting image data displayed by the displaying step onto the image forming material.

In the image forming method, it is preferable that the image forming method comprises checking the final image data whether a desired image has been obtained, correcting the outputting image data in accordance with a result of the checking step, outputting the corrected image data onto the image forming material, and conducting the post-processing to the image forming material.

In the image forming method, it is preferable that the plural image forming materials each have a support having thereon a image forming layer, which contains a photosensitive silver halide, a photo-insensitive organic silver salt and a reducing agent, and a protective layer. Further, it is more preferable that the plural image forming materials each have an intermediate layer, the image forming layer and the protective layer in that order on the support. It is also preferable that the plural image forming materials each have the image forming layer, a barrier layer and the protective layer in that order on the support.

A third structure of the invention is an image forming system. The image forming system comprises:

a medical image data-inputting apparatus including a medical image data-sending section;

a medical image data-managing apparatus including an image data-storing section and a medical image data-transferring section;

an image data-converting apparatus including an image forming material-selecting section, an image data-converting section and an outputting image data-transferring section; and

an outputting apparatus including a first image forming material-supplying section supplying a first image forming material, second image forming material-supplying section supplying a second image forming material being different from the first image forming material, an outputting section and a post-processing section.

The inputting apparatus, the medical image data-managing apparatus, the image data-converting apparatus and the outputting apparatus is connected via a network.

In the image forming system, the medical image data-inputting apparatus sends a digitalized medical image data by the medical image data-sending section to the medical image data-managing apparatus. The medical image data-managing apparatus stores the medical image data in the image data-storing section, and transfers a medical image data to be output from the image data-storing section to the image data-converting apparatus. The image data-converting apparatus selects an image forming material to be output from the first and second image forming materials by the selecting section, converts the transferred medical image data to an outputting image data being suited to the selected image forming material by the converting section; and transfers the outputting image data with a result of the selection in the selecting section to the outputting apparatus by the outputting image-transferring section. Subsequently, the outputting apparatus outputs the outputting image data onto the selected image forming material, which is supplied from the first or second image forming material-supplying section in accordance with the result of selection, by the outputting section; and conducts a post-processing to the selected image forming material to form a final image.

In the image forming system, it is preferable that the converting section has at least one of a resolution-converting function, a gradient-converting function, a color-converting function and an LUT-converting function.

In the image forming system, it is preferable that the medical image data-inputting apparatus is a medical image diagnosis apparatus.

In the image forming system, it is preferable that the image forming system comprises two or more of the medical image data-inputting apparatus connected via the network.

### BRIEF DESCRIPTION OF THE DRAWINGS

FIG. 1 is a flow chart of the structures of the image forming apparatus of the present invention.

FIG. 2 is a flow chart of the structures of another image forming apparatus of the present invention.

FIG. 3 is a flow chart of the structures of still another image forming apparatus of the present invention.

FIG. 4 is a flow chart showing a case in which a medical image database managing apparatus, which manages and stores digitalized medical image data, and an image forming apparatus are connected via a network.

FIG. 5 is a schematic view showing an image forming apparatus capable of forming images onto three different image forming materials.

FIG. 6 is a schematic view showing another image recording apparatus capable of forming images onto three different image forming materials.

FIG. 7 is an example of a flow chart showing a network related to medical image data in a medical organization.

FIG. 8 is a flow chart showing a part of the medical image forming system of the invention.

FIG. 9 is a flow chart showing a structure including a display apparatus correction section, which carries out graduation correction.

FIG. 10 is a flow chart showing a part of another example of the medical image forming system of the invention.

### DETAILED DESCRIPTION OF THE INVENTION

The present invention will now be detailed. Initially an image forming material employed in the image forming apparatus, image forming method and image forming system of the present invention will be detailed.

Depending on the use, appropriately selected and used as supports of the image forming material suitably employed in the image forming apparatus, image forming method and image forming system of the present invention may be the transparent supports or reflective supports detailed below.

Examples of resins employed to form supports which are employed to prepare the image forming materials employed in the present invention include acryl based resins, polyester, polycarbonate, polyarylate, polyvinyl chloride, polyethylene, polypropylene, polystyrene, nylon, aromatic polyamides, polyether ether ketone, polysulfone, polyether sulfone, polyimide, polyether imide, and triacetylcellulose. These are employed to form sheets of film, which may be prepared by laminating at least two layers of these resins.

From the aspect of dimensional stability, preferred as such supports are those which are stretched into a film and then annealed. Of the aforesaid resins, preferred are, for example, polyester, polycarbonate, polyarylate, polyether ketone, and triacetylcellulose. From the viewpoint of general utility and/or cost, polyester which is biaxially oriented and annealed is further preferred.

The aforesaid polyester will now be detailed. Polyester, as described herein, refers to a high-molecular compound having an ester linkage in the main chain of the molecule, and to a polymer which is prepared while undergoing condensation polymerization of diol and dicarboxylic acid. Dicarboxylic acids, as descried herein, are those represented by terephthalic acid, isophthalic acid, phthalic acid, naphthalenedicarboxylic acid, adipic acid, and sebacic acid. Further, diols, as described herein, are those represented by ethylene glycol, trimethylene glycol, tetramethylene glycol, and cyclohexanedimethanol. Of these, preferably employed are polymethine terephthalate (PET) or its copolymers, polybutylene naphthalate (PBN) or its copolymers, polybutylene terephthalate (PBT) or its copolymers, and polyethylene naphthalate (PRN) or its copolymers. The number of related units of these polyesters is preferably at least 100 and is more preferably at least 150. The intrinsic viscosity is preferably at least 0.6 dl/g, and is more preferably at least 0.7 dl/g. Such cases are preferred due to excellent film forming stability. In order to prepare desired transparent or reflective supports, incorporated optionally into these polyesters may be obviously prior art additives such as lubricants, stabilizers, antioxidants, viscosity modifiers, antistatic agents, coloring agents, and pigments.

Further, when reflective supports are prepared, it is preferable that the aforesaid supports be made white by the addition of coloring agents. Listed as such white pigments may be fillers such as titanium oxide, zinc oxide, barium sulfate, and calcium carbonate. Further, it is possible to preferably use porous supports which are prepared in such a manner that film is prepared to form pours in the interior during biaxial orientation by adding resins such as polypropylene which are incompatible with the aforesaid fillers or polyester.

Alternatively, blue dyes may be incorporated into transparent supports. Examples of such dyes include disperse dyes, cationic dyes, basic dyes, acid dyes, reactive dyes, direct dyes, vat dyes, azoic dyes, mordant dyes, acid mordant dyes, union dyes, and solvent dyes. These dyes may be suitably selected and then employed. Of the aforesaid dyes, from the viewpoint of the uniform dispersibility during melt-kneading in preparation of the support and the dye solubility during the preparation of the coating composition to form the backing layer described below, solvent dyes are preferred. Further, for the purpose of minimizing sublimation of dyes during melt-kneading as well as minimizing modification of dyes during kneading, dyes are preferred which exhibit heat resistance to at least 250° C. Further, when the temperature of an extrusion apparatus which is employed to extrude and cast resins which are employed to prepare the support requires to increase at least 300° C. or more to function properly dyes which exhibit heat resistance of 280° C. or higher are more preferred. Further, for the purpose of resulting in blue coloring, dyes of a λmax of 600-650 nm are preferred.

Still further, the thickness of the aforesaid supports is customarily 50-500 μm, and is preferably 100-250 μm.

Further, to improve conveying properties, as well as antistatic properties and antihalation properties, a backing layer may be provided on the surface opposite the surface of the support onto which the image forming layer is applied. The aforesaid backing layer is comprised of binder resins and various additives which are added, if desired.

Binder resins which form the backing layer may be selected from transparent or translucent binder resins which are conventionally used, and subsequently employed. Examples of such binder resins include polyvinyl acetal based resins such as polyvinyl formal, polyvinyl acetal, and polyvinyl butyral, cellulose based resins such as nitrocellulose, cellulose acetate propionate, and cellulose acetate butyrate, styrene based resins such as polystyrene, styrene-acrylonitrile copolymers, and styrene-acrylonitrile-acryl rubber copolymers, vinyl chloride based resins such as polyvinyl chloride and vinyl chloride-vinyl acetate copolymers, acryl based resins such as polymethyl methacrylate, polyester resins, polyurethane resins, polycarbonate resins, polyacrylate resins, epoxy resins, phenoxy resins, aromatic polyester resins, and the aforesaid resin modified products. Further, employed as layer forming binders may be epoxy group containing compounds and acryl group containing compounds on the premise that the layer is cured while exposed to actinic radiation. Further, these binders may be employed individually or in combinations of at least two types of resins.

Further, when the aforesaid binder resins comprise a hydroxyl group, crosslinking agents such as metal alkoxides which have, in the molecule, a plurality of metal alkoxide portions such as multifunctional isocyanate compounds, alkoxysilane compounds, and alkoxytitanium compounds, which are conventionally known in the art, may be incorporated to undergo crosslinking.

As other various types of additives, for the purpose of minimizing insufficient pick-up and assuring conveyance properties, fillers are preferably incorporated. Specific examples of fillers include inorganic fillers such as SiO₂, TiO₂, BaSO₄, ZnS, MgCO₃, CaCO₃, ZnO, CuO, Ca, WS₂, MoS₂, MgO, SnO₂, Al₂O₃, α-Fe₂O₃, α-FeO₂H, SiC, CeO₂, Y₂O₃, ZrO₂, MoC, BC, WC, BN, SiN, titanium carbide, corundum, artificial diamond, garnet, quartzite, tripoli, diatomaceous earth, and dolomite, as well as organic fillers such as polyethylene resin particles, fluorine resin particles, guanamine resin particles, acrylic resin particles, silicone resin particles, melamine resin particles, and silk powder. The proportion of added fillers is preferably 0.05-30 percent by weight with respect to the backing layer forming compositions.

Further, in order to improve lubrication properties as well as antistatic properties, incorporated into the backing layer may be lubricants and antistatic agents. Examples of such lubricants include fatty acids, fatty acid esters, fatty acid amides, polyoxyethylene, polyoxypropylene, silicone oil (modified), silicone resins (modified), fluorine compounds (modified), fluorine resins (modified), carbon fluoride and wax. Still further, listed as antistatic agents may be cation based surfactants, anion based surfactants, nonionic surfactants, polymer antistatic agents, metal oxides, or conductive polymers described in U.S. Pat. No. 5,747,412, and compounds described on pages 875 and 876 of “11290 no Kagaku Shohin (11290 Chemical Products)”, Kagakukogyo Nippo Sha.

The thickness of the backing layer is customarily about 0.5-about 10 μm, and is preferably 1.0-5 μm. Further, the backing layer may be comprised of a single layer or a plurality of layers which are comprised of different compositions.

Further, for the purpose of minimizing static charge, an antistatic layer may be provided between the support and the backing layer. In addition, in order to improve adhesion property as well as coatability, the support surface onto which the backing layer is applied may be modified employing prior art surface modifying techniques such as a corona discharge treatment, a plasma treatment, or an anchor coat treatment.

In the invention, an image forming material having an image forming layer containing, as essential components, photosensitive silver halide, non-photosensitive organic silver and reducing agents, is preferably used. In the present embodiment, other than the aforesaid essential components, binder resins and, if desired, various additives may further be incorporated.

In order to minimize milky whiteness after image formation, and ensure excellent image quality, it is preferable that the average grain size of light-sensitive silver halide, added as an essential component, is as small as possible. The average grain size is preferably at most 0.1 μm, and is more preferably 0.01-0.1 μm. The grain size, as described herein, refers to the diameter (circle equivalent diameter) of a circle having the same area as each of the particles observed by an electron microscope. Further, the silver halide is preferably monodispersed and is more preferably at most 30 percent. Monodispersion, as described herein, refers to dispersion in which the degree of monodispersion obtained by the formula described below is at most 40 percent.

Degree of Monodispersion (percent)=(standard deviation of the particle diameter)/(average of the particle diameter)×100

The shape of light-sensitive slier halide grains is not particularly limited. However, it is preferable that the ratio, which is occupied by the Miller index [100] plane, is greater. The aforesaid ratio is preferably at least 50 percent, and is more preferably at least 70 percent. It is possible to obtain the ratio of the Miller index [100] plane based on T. Tani, J. Imaging Sci., 29, 165 (1985), which utilizes adsorption dependence of a [111] plane and a [100] plane in sensitizing dye adsorption.

Further, the shape of light-sensitive silver halide may be tabular. A tabular grain, as described herein, refers to a grain having an aspect ratio (r/h) of at least 3, wherein r (in μm) represents the grain diameter which is the square root of the projected area, and h (in μm) represents the thickness in the vertical direction. Grains having an aspect ratio of 3-50 are preferred. Further, the grain diameter is preferably at most 0.1 μm, and is more preferably 0.01-0.08 μm. Tabular grains are described in U.S. Pat. Nos. 5,264,337, 5,314,789, and 5,320,958. These targeted tabular grains are easily prepared.

The composition of light-sensitive silver halide is not particularly limited, and any compound of silver chloride, silver chlorobromide, silver iodobromide, silver bromide, silver iodobromide, and silver iodide may be employed. Emulsions employed in the present invention may be prepared employing the methods described in P. Glafkides, Chimie et Physique Photographique (published by Paul Montel Co., 1957); G. F. Duffin, Photographic Emulsion Chemistry (published by The Focal Press, 1966); and V. L. Zelikman et al, Making and Coating Photographic Emulsion (published by The Focal Press, 1964).

Further, metal ions which belong to Groups 6-11 in the Periodic Table are preferably incorporated into light-sensitive silver halide. Listed as such metals may be tungsten, iron, cobalt, nickel, copper, ruthenium, rhodium, palladium, rhenium, osmium, iridium, platinum, and gold. These metals may be incorporated into silver halide in the form of a metal complex or a metal complex ion.

Preferred as the aforesaid metal complexes or metal complex ions are the six-coordinate metal complexes represented by General Formula (5) described below.

[ML₆]^{m} General Formula (5)

In General Formula (5), M represents a transition metal selected from the elements in Groups 6-11 in the Periodic Table, L represents a ligand, and m represents 0, -, 2-, 3-, or 4-. Specific examples of ligands represented by L include ligands such as halides (for example, fluoride, chloride, bromide, and iodide), cyanide, cyanato, thiocyanato, selenocyanato, tellurocyanato, azido, and aquo. When an aquo ligand is present, it is preferable that one or two ligands are occupied by the aquo ligand. L may be the same or different.

Further, M in General Formula (5) is preferably copper, iron, rhodium, ruthenium, rhenium, iridium, or osmium.

Incidentally, the aforesaid metal ions, metal complexes, and metal complex ions may be employed individually or in combinations of at least two types. The proportion of these metal ions, metal complexes, and metal ions is suitably 1×10⁻⁹-1×10⁻² mol per mol of light-sensitive silver halide and is preferably 1×10⁻⁸-1×10⁻⁴ mol.

It is preferable that compounds which provide these metals are added during formation of silver halide grains and are incorporated into the light-sensitive silver halide grains. The aforesaid compounds may be added during any stage of the preparation of light-sensitive silver halide grains, i.e., nuclei formation, growth, physical ripening, and prior to or after chemical ripening. Specifically, however, the aforesaid compounds are preferably added during the stages of nuclei formation, growth, and physical ripening, and more preferably added during the stage of nuclei formation and growth.

When added, the aforesaid compounds may be divided into several portions and added at optional intervals, whereby it is possible to incorporate them uniformly in the light-sensitive grain. On the other hand, it is also possible to incorporate them to result in desired distribution in the grain as described in JP-A Nos. 63-29603, 2-306236, 3-167545, 4-76534, 6-110146, and 5-273683.

It is possible to add these metal compounds while dissolved in water or suitable organic solvents (such as alcohols, ethers, glycols, ketones, or esters), employing any of the following methods; a method in which an aqueous solution prepared by dissolving metal compound powder in water, or an aqueous solution prepared by dissolving metal compounds together with sodium chloride, or potassium chloride in water is added to a solution of water-soluble silver salts or a solution of water-soluble halide which is employed to form grains, a method in which silver halide grains are formed employing a triple-jet method in such a manner that when a silver salt solution and a halide solution are mixed employing a double-jet method, a metal compound solution is used as a third solution, a method in which during formation of grains, an aqueous solution of metal compounds in a necessary amount is charged into a reaction vessel, or a method in which during formation of light-sensitive silver halide, other light-sensitive silver halide grains which have been doped with metal ions or complex ions are added and dissolved. It is possible to employ a suitable method from any of these methods. Further, when added to the surface of grains, it is possible to charge an aqueous solution of metal compounds in a necessary amount into a reaction vessel immediately after grain formation, during physical ripening or at its completion, or during chemical ripening.

Light-sensitive silver halide grains may be or may not be desalted after grain formation. When desalted, it is possible to carry out desalting employing methods known in the industry, such as a noodle method, a flocculation method, etc.

If desired, light-sensitive silver halide grains employed in the present invention may be subjected to chemical sensitization. When subjected to such chemical sensitization, it is possible to use any of the methods well known in the industry, such as a sulfur sensitization method, a selenium sensitization method, or a tellurium sensitization method, each as appropriate. Further, it is possible to use a noble metal sensitization method in which gold, platinum, palladium, or iridium compounds are used, as well as a reduction sensitization method.

Light-insensitive organic silver salts incorporated into the image forming layer as an essential component are silver salts of organic acids. Organic acids employed to form the aforesaid organic silver salts include aliphatic carboxylic acids, carbon cyclic carboxylic acids, heterocyclic carboxylic acids, and heterocyclic compounds. Of these preferred are long chain (having 10-30 carbon atoms and preferably 15-25 carbon atoms) aliphatic carboxylic acids and heterocyclic carboxylic acids having a nitrogen-containing heterocyclic ring. Further organic silver salt complexes in which the ligand has a total stability constant of 4.0-10.0 with respect to silver ions may be employed as appropriate.

Examples of such organic silver salts are described in Research Disclosure (hereinafter referred to as RD) Items 17029 and 29963; British Patent No. 1,439,478; Japanese Patent Application Open to Public Inspection Nos. 10-236004, 2000-62325, and 2002-23303; and European Patent Nos. 962,815 and 964,300. Of these, aliphatic acid silver salts are preferably employed. Of aliphatic silver salts, most preferably employed are silver behenate, silver arachidate, and silver stearate.

Further, in order to effectively exhibit the desirable effects of the present invention, of the aforesaid three aliphatic acids, the proportion of silver behenate having a higher melting point of the parent aliphatic acid is preferably at least 40 percent by weight and more preferably at least 60 percent by weight.

Still further, the average grain diameter of organic silver salts is at most 1 μm and the aforesaid salts are monodispersed. The average grain diameter, as described herein, refers to the diameter of a sphere having the same volume as the organic silver grain when the organic silver salt grain is a spherical grain, a bar-shaped grain, or a tabular grain. The average grain diameter is customarily 0.01-0.8 μm, but is preferably 0.05-0.5 μm. Further, monodispersion, as described herein, is as defined for silver halide grains and the degree of monodispersion is preferably 1-30 percent. In the present embodiment, it is preferable that organic silver salts result in monodispersed grains of an average grain diameter of at most 1 μm. By adjusting the average grain diameter to the aforesaid range, it is possible to prepare higher density images. It is preferable that organic silver salts are comprised of at least 60 percent tabular grains with respect to all organic silver salts. Tabular grains of organic silver salts, as described in the present invention, are as defined for the tabular grains of the aforesaid light-sensitive silver halide and refer to those of an aspect ratio of at least 3.

It is preferable that such organic silver grains are prepared in such a manner that, if desired after achieving preliminary dispersion in the presence of surfactants, the resulting grains are dispersed and pulverized employing a media homogenizer or a high pressure homogenizer. In the aforesaid preliminary dispersion, employed may be common stirrers such as an anchor type or a propeller type, a high speed rotation centrifugal radial stirrer (a dissolver), or a high speed rotation shearing type stirrer (a homomixer). Further, employed as the aforesaid media homogenizers may be tumbling mills such as a ball mill, a planet ball mill, or a vibration ball mill, a bead mill which is a media stirring mill, an attritor, and a basket mill. Employed as high pressure homogenizers may be various types such as a type in which dispersion is carried out while allowing a composition to collide with walls or plugs, a type in which a composition is divided into a plurality of portions and divided compositions are collided with each other at high speed, or a type in which a composition is passed through narrow orifice(s). Further, suitably selected and employed as media for dispersion may be ceramics such as zirconia, alumina, silicon nitride, boron nitride, or diamond.

Suitably selected and employed as reducing agents incorporated, as a essential component, into the image forming layer of the image forming material of the present invention are phenols, polyphenols having at least two hydroxyl groups, naphthols, bisnaphthols, polyhydroxybenzenes having at least two hydroxyl groups, polyhydroxynaphthalenes having at least two hydroxyl groups, ascorbic acids, 3-pyrazolidones, pyrazolone-5-ones, pyrazolines, phenylenediamines, hydroxyamines, hydroquinone monoethers, hydroxamic acids, hydrazides, amidoximes, and N-hydroxyureas.

Of the aforesaid reducing agents, listed as preferred reducing agents, when aliphatic carboxylic acid silver salts are employed as an organic silver salt, may be polyphenols in which at least two phenol groups are linked via an alkylene group or via sulfur, and specifically polyphenols in which at least two phenol groups, in which an alkyl group (e.g., a methyl group, an ethyl group, a propyl group, a t-butyl group, or a cyclohexyl group) or an acyl group (e.g., an acetyl group or a propionyl group) is substituted at one or more positions adjacent to the hydroxyl substituted position of the aforesaid phenol group, and are linked via an alkylene group or via sulfur. Examples of such compounds include 1,1-bis(2-hydroxy-3,5-dimethylphenyl)-3,5,5-trimethylhexane, 1,1-bis(2-hydroxy-3-t-butyl-5-methylphenyl)methane, 1,1-bis(2-hydroxy-3,5-di-t-butyl-5-methylphenyl)methane, 1,1-bis(2-hydroxy-3-methyl-5-t-butylphenyl)methane, 1,1-bis[2-hydroxy-3-methyl-5-(1-methylhexyl)phenyl]methane, (2-hydroxy-3-t-butyl-5-methylphenyl)-(2-hydroxy-5-methylphenyl)methane, 6,6′-benzylidene-bis(2,4-di-t-butylphnol), 6,6′-benzylidene-bis(2-t-butyl-4-methylphnol), 6,6′-benzylidene-bis(2,4-dimethylphnol), 1,1-bis(2-hydroxy-3,5-dimethylphenyl)-2-methylpropane, 1,1-bis(2-hydroxy-3,5-dimethylphenyl)-1-cyclohexylmethane, 1,1-bis(2-hydroxy-3,5-dimethylphenyl)-1-(2,4-dimethyl-3-cyclohexenyl)methane, 1,1-bis(2-hydroxy-3,5-dimethylphenyl)-1-(2-methyl-4-cyclohexenyl)methane, 1,1-bis(2-hydroxy-3,5-dimethylphenyl)-1-(2-methyl-4-cyclohexyl)methane, 1,1,5,5-tetrakis(2-hydroxy-3,5-dimethylphenyl)-2,4-ethylpentane, 2,2-bis(4-hydroxy-3,5-dimethylphenyl)propane, and 2,2-bis(4-hydroxy-3,5-di-t-butylphenyl)propane. Further, listed may be bisnaphthols described in U.S. Pat. Nos. 3,589,903 and 4,021,249; British Patent No. 1,486,148; JP-A Nos. 51-51933, 50-36110, 50-116023, 52-84727, 2001-56527, 2001-42469, 2001-92075, and 2001-188323; and Japanese Patent Publication No. 51-35727, which include, for example, 2,2′-dihydroxy-1,1′-binaphthyl, 6,6′-dibromo-2,2′-dihydroxy-1,1′-binaphthyl, 6,6′-dinitro-2,2′-dihydroxy-1,1′-binaphthyl, bis(2-hydroxy-1-naphthyl)methane, 4,4′-dimethoxy-1,1′-dihydroxy-2,2′binaphthyl. Still further, listed may be sulfonamidophenols or sulfonamidonaphthols, described in U.S. Pat. No. 3,801,321, which include, for example, 4-benzenesulfonamidophenol, 2-benzenesulfonamidophenol, 2,6-dichloro-4-benzenesulfoamidophenol, and 4-benzenesulfoamidophenol.

The amount of reducing agents incorporated into the image forming layer of the present invention varies depending on the types of light-insensitive organic silver salts and reducing agents, as well as other additives described below. However, the aforesaid amount is customarily 0.05-10 mol with respect to mol of light-insensitive organic silver salts, and is preferably 0.1-3 mol. Further, in the above addition amount range, the aforesaid reducing agents may be employed in combinations of at least two types.

In the present invention, in order to retain the aforesaid essential components, employed are binder resins in the image forming layer. Suitably selected and employed as such binder resins may be those detailed in the aforesaid backing layer in an amount range which does not adversely affect the purposes of the present invention. However, since it is necessary to allow the aforesaid light-sensitive silver halide, light-insensitive organic salvers salts, and reducing agents to be dispersed and retained, employing the binder resins, resin are preferred which have in the molecule, a hydroxyl group or a carboxyl group, or salts thereof, or sulfonic acid or salts thereof. Listed as such resins are polyvinyl acetal based resins, cellulose based resins, phenoxy resins, aromatic polyester resins, further modified vinyl chloride based resins, modified polyesters, modified polyurethanes, modified epoxy resins, and modified acryl based resins, which are prepared by introducing the aforesaid functional groups. Theses resins may be employed individually or in combinations of at least two types.

Further, when the aforesaid binder resins have a hydroxyl group or active hydrogen, layer strength may be enhanced by adding crosslinking agents known in the art such as metal alkoxides which have in the molecule, a plurality of metal alkoxide portion, such as conventional multifunctional isocyanate compounds, alkoxysilane compounds, or alkoxytitanium compounds.

Further, other than the aforesaid essential components, binder resins, and crosslinking agents, incorporated if desired, added to the image forming layer of the image forming material of the present invention may be color tone control agents, silver saving agents, antifoggants, toning agents, sensitizing dyes, and supersensitization exhibiting materials (hereinafter referred to as supersensitizers). Color tone control agents, as described herein, refer to compounds which result through their addition in special absorption variation in the spectral absorption of the resulting images. It is possible to form image forming materials which result in different tones which are employed in the image forming apparatus of the present invention under the presence or absence of the aforesaid agents or by varying the added amount.

Further, silver saving agents, as described herein, refer to compounds capable of decreasing the silver amount, which is necessary to result in a definite silver image density. Even though several action mechanisms are considered to describe a silver decreasing function, preferred are compounds which exhibit functions to enhance the covering power of developed silver. The covering power of developed silver, as described herein, refers to the optical density with respect to unit weight of silver. By adding or not adding such silver saving agents or varying the added amount, it is possible to form image forming materials which result in a difference in the maximum density of images, which are employed in the image forming apparatus of the present invention.

Appropriately selected as color tone control agents may be those described in JP-A Nos. 10-268465 and 10-228076 in which color tone control agents are incorporated into microcapsules. It is also possible to select methods, in which couplers described in JP-A Nos. 11-288057, 2001-330923, 2001-330925, 2001-264926, 2001-264927, 2001-264928, and 2002-49123, are employed to undergo color formation. Alternatively, it is preferable to utilize leuco coloration described in JP-A No. 11-231460.

Of such leuco dyes, preferred as compounds which result in special spectral absorption in the yellow-magenta region are compounds represented by the following General Formula (1).

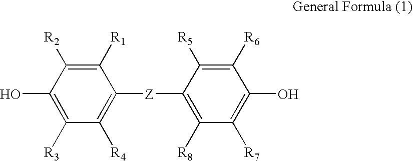

In the bisphenol compounds represented by General Formula (1), examples of substituents represented by each of R₁-R₈, and, R₉ and R₁₀ include an alkyl group (such a methyl, ethyl, propyl, isopropyl, cyclopropyl, butyl, isobutyl, sec-butyl, t-butyl, cyclohexyl, or 1-methyl-cyclohexyl group), an alkenyl group (such a vinyl, propenyl, butenyl, pentenyl, isohexenyl, cyclohexenyl, butenylidene, or isopentylidene group), an alkynyl group (such an ethynyl or propinylidne group), an aryl group (such a phenyl or naphthyl group), a heterocyclic group (such a furyl, thienyl, pyridyl, or tetrahydrofuranyl group), and others such as a halogen group, a hydroxyl group, an alkoxy group, an aryloxy group, an acyloxy group, a sulfonyloxy group, a nitro group, an amino group, an acylamino group, sulfonylamino group, a sulfonyl group, a carboxy group, an alkoxycarbonyl group, an aryloxycarbonyl group, a carbamoyl group, a sulfamoyl group, a cyano group, and a sulfo group. These substituents may be substituted with another substituent previously described.

Of these, substituents represented by R₉ or R₁₀ are preferably a halogen atom or a substituted or unsubstituted alkyl group, while R₁-R₈ each independently is preferably a hydrogen atom, a substituted or unsubstituted alkyl group, an alkenyl group, an alkynyl group, an aryl group, or a heterocyclic group.

Specific examples of bisphenol compounds, represented by General Formula (1), will be described below. However, the present invention is not limited thereto.

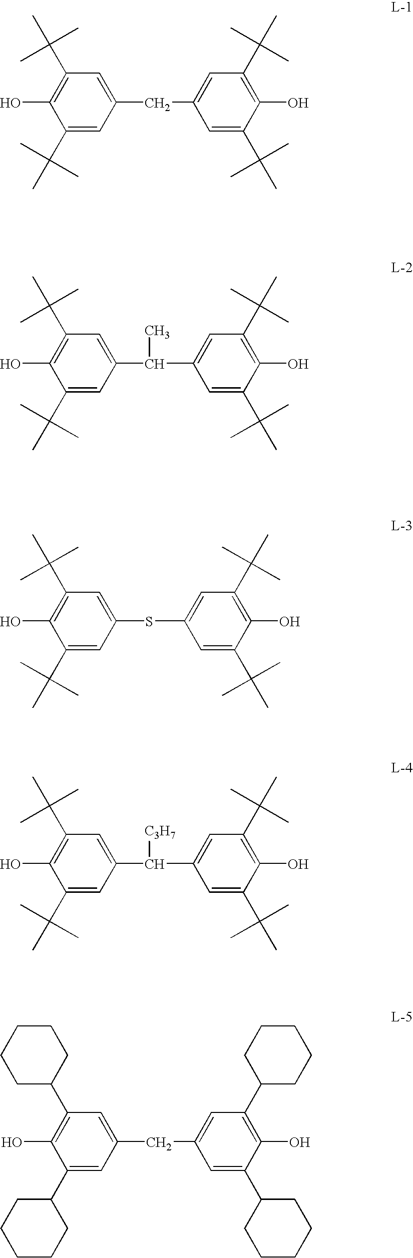

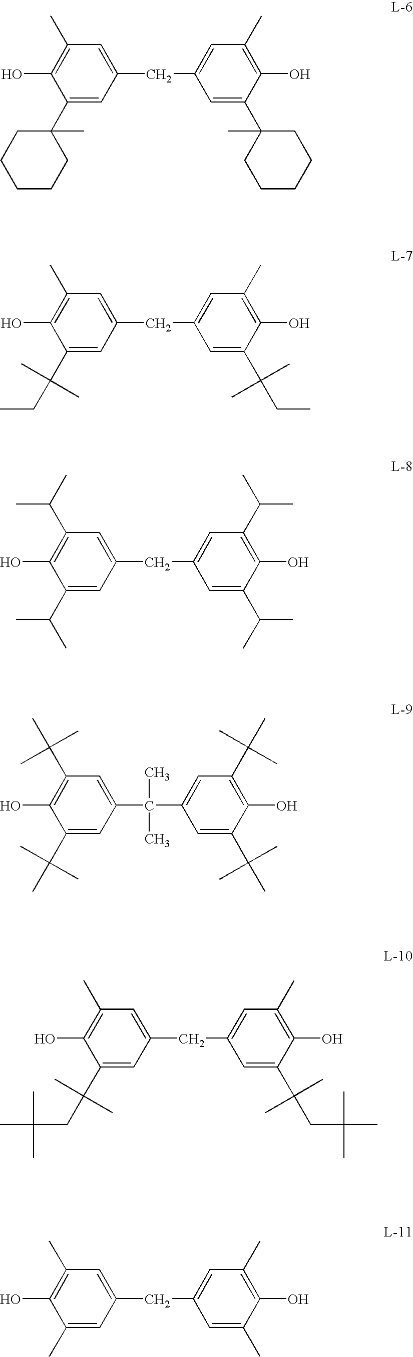

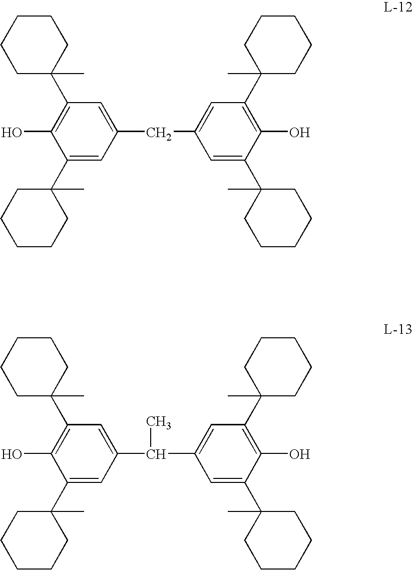

The used amount of the compounds represented by General Formula (1) is suitably in the range of 0.001-10 mol per mol of silver, and is preferably 0.002-1.0 mol. Further, in the aforesaid added amount range, the aforesaid color tone control agents may be employed in combinations of at least two types and may also be incorporated into a coated protective layer, a barrier layer or an interlayer, described below, other than the image forming layer.

Listed as silver saving agents employed in the present invention are hydrazine compounds and vinyl compounds, which are described in U.S. Pat. Nos. 5,496,695, 5,545,505, 5,545,507, 5,637,449, 5,654,130, 5,635,339, 5,545,515, and 5,686,228, and JP-A Nos. 10-339928, 11-84576, 11-95365, 11-95366, 11-109546, 11-119372, 11-119373, 2000-356834, 2001-27790, and 2001-174947.

Further, preferably employed as silver saving agents may be Schiff bases which are prepared by dehydration condensation reaction of alkoxysilane compounds having at least two primary or secondary amino groups or salts thereof, and/or alkoxysilane compounds having at least one primary amino group along with ketone compounds. “Having at least two primary or secondary amino groups” refers to having at least only two primary amino groups, having at least two secondary amino groups, having at least one primary amino group as well as at least one secondary amino group. The salts of alkoxysilane compounds, as described herein, refer to addition products of inorganic or organic acids, which are capable of forming onium salts with an amino group, with alkoxysilane compounds.

Listed as such alkoxysilane compounds or salts thereof and Schiff bases may be those described below. However, these compounds are not limited as long as they are alkoxysilane compounds having at least two primary or secondary amino groups in the molecule or salts thereof, and/or Schiff bases which are formed by dehydration condensation reaction of alkoxysilane compounds having at least one primary amino group along with ketone compounds.

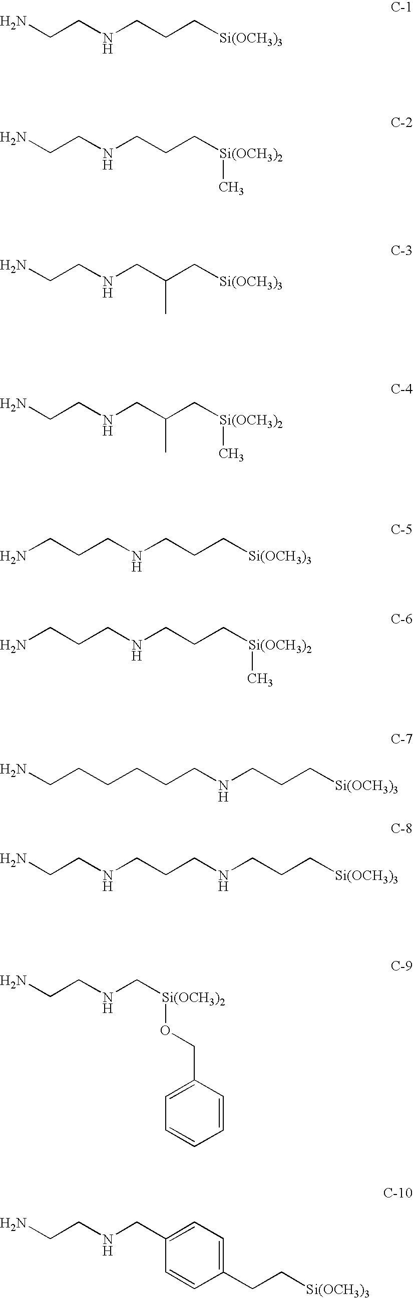

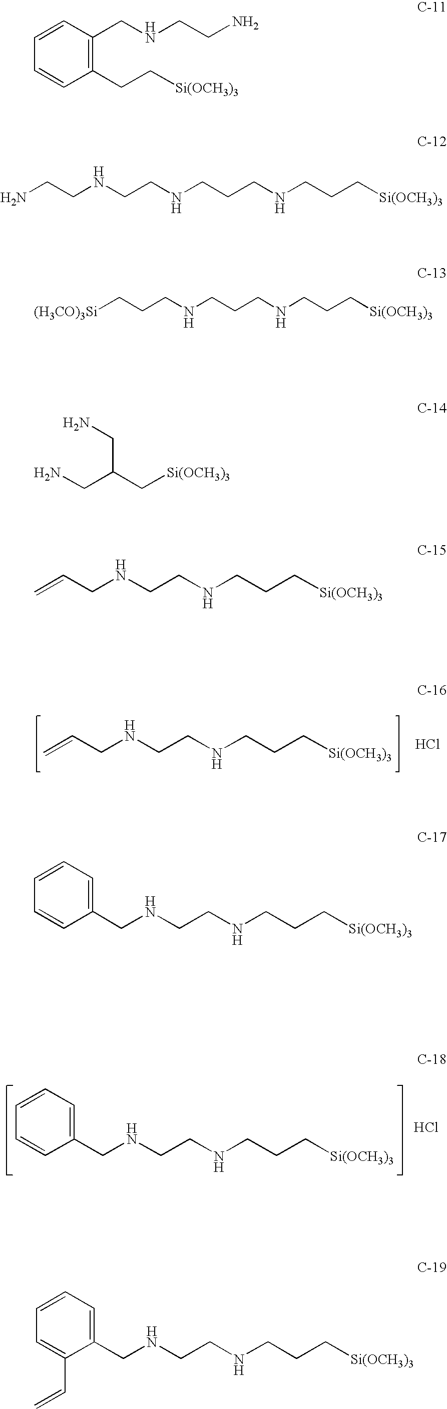

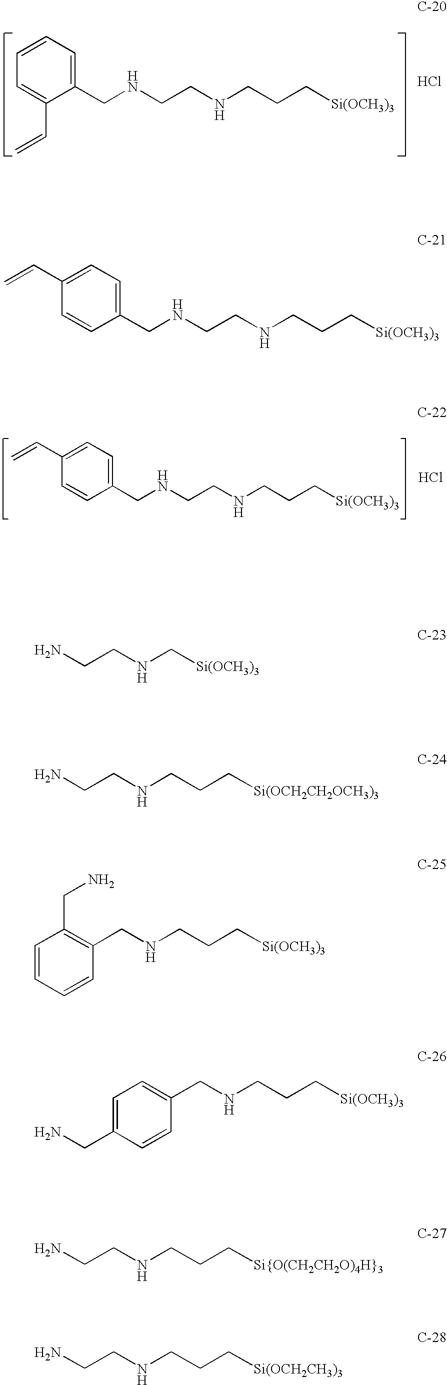

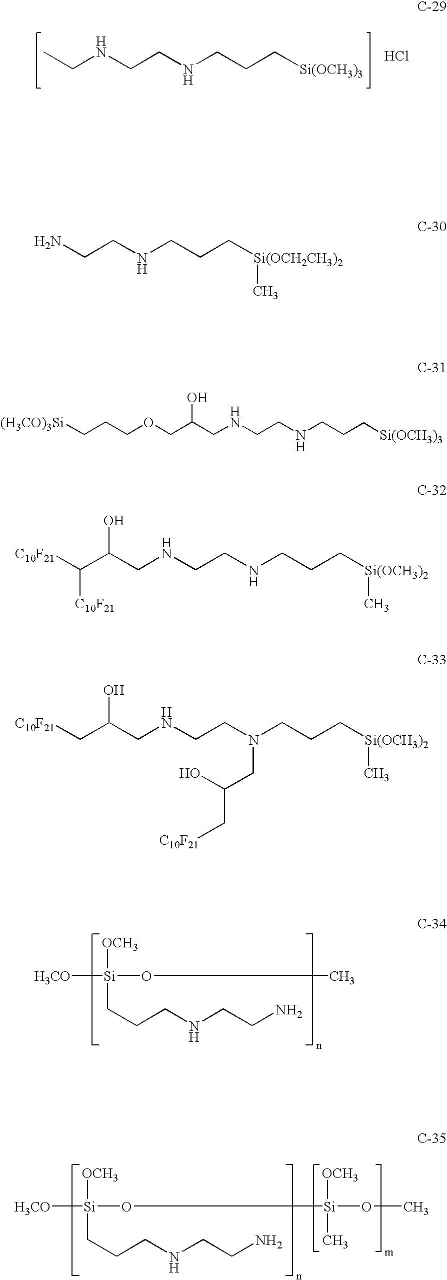

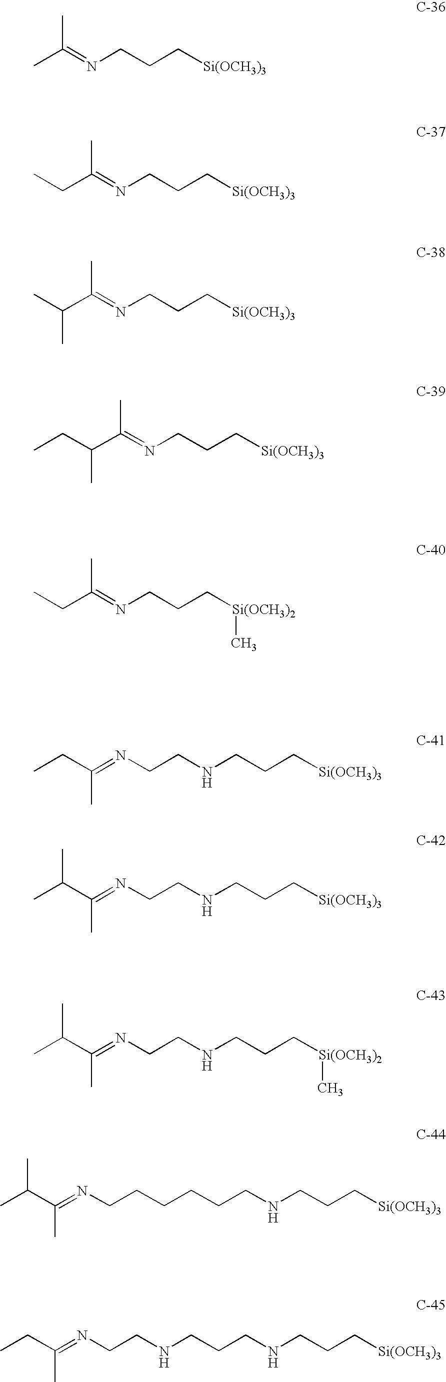

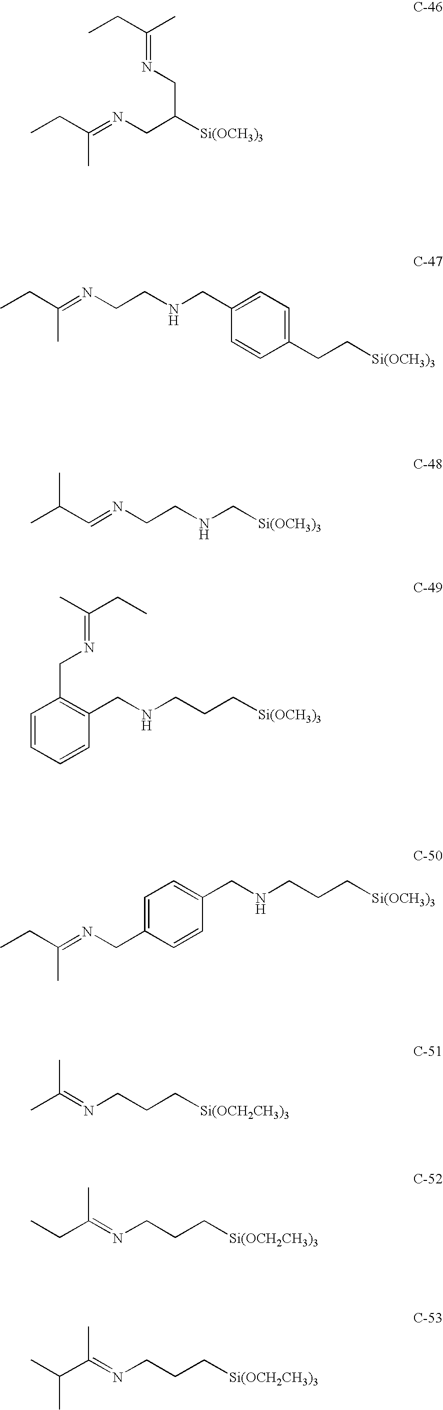

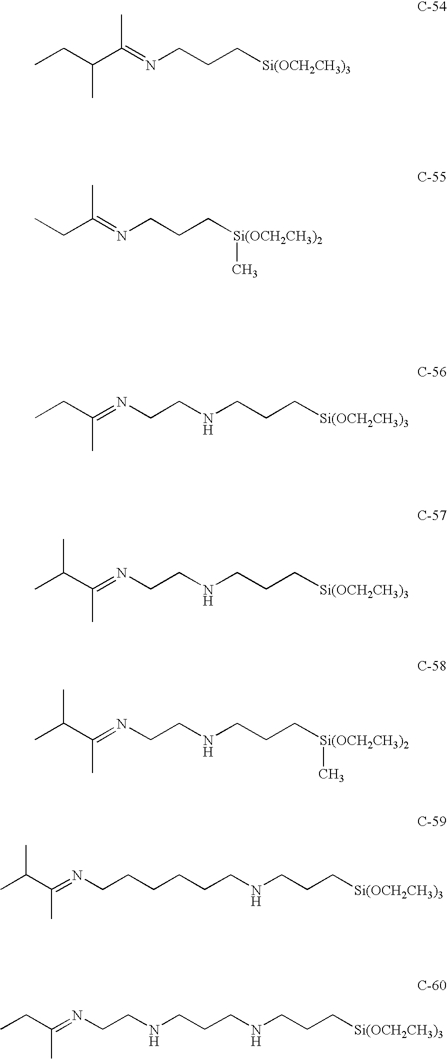

In the aforesaid compounds, preferred as alkoxy groups which form alkoxysilyl are alkoxy groups comprised of saturated hydrocarbon. Further, a methoxy group, an ethoxy group, and an isopropoxy group are preferred to enhance storage stability. Further, the added mount of these alkoxysilane compounds or salts thereof, or Schiff bases in the image forming layer is preferably in the range of 0.00001-0.05 mol per mol of silver and they may be incorporated into the protective layer, barrier layer, and interlayer described below.

Preferred as antifoggants, which are incorporated into the image forming layer for the purpose of improving storage stability as well as image retaining properties, are halogen compounds represented by General Formula (2), described below.

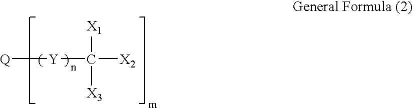

In General Formula (2), X₁, X₂, and X₃ each represents a hydrogen atom, a halogen atom, an acyl group, an alkoxycarbonyl group, an aryloxycarbonyl group, a sulfonyl group, or an aryl group, and at least one of them represents a halogen atom.

Y represents —NR—C(═O)—, —C(═O)—, —Z—C(═O)—, —Z—S(═O)—, —SO— or —SO₂—, n represent an integer of 0-2, and m represents an integer of 1-10. Further, R represents a hydrogen atom, or an alkyl group, and may form a ring structure with Q, described below, while Z represents an oxygen atom or a sulfur atom.

In General Formula (2), Q represents an alkyl group, an aryl group, or a heterocyclic group, and any of these groups may have a substituent. Alkyl groups, as represented by Q in General Formula (2), include a straight chain, branched chain or cyclic alkyl group having preferably 1-30 carbon atoms, and more preferably 1-20 carbon atoms. The alkyl group represented by Q may have a substituent. Employed as such substituents may be any groups as long as micro-encapsulation is not adversely affected. Examples of such substituents include a halogen atom, an alkenyl group, an alkynyl group, an aryl group, a heterocyclic group (including an N-substituted nitrogen containing heterocyclic group such as a morpholino group), an alkoxycarbonyl group, an aryloxycarbonyl group, a carbamoyl group, an imino group, an imino group substituted with a nitrogen atom, a thiocarbonyl group, a carbazoyl group, a cyano group, a thiocarbamoyl group, an aryloxy group, a heterocyclicoxy group, an acyloxy group, an (alkoxy or aryloxy)carbonyloxy group, a sulfinyloxy group, an acylamido group, a sulfonamido group, a ureido group, a thioureido group, an amido group, an (alkoxy or aryloxy)carbonylamino group, a sulfamoylamino group, a semicarbazido group, an (alkyl or aryl)sulfonylureido group, a nitro group, an (alkyl or aryl)sulfinyl group, a sulfamoyl group, a group containing phosphoric acid amido or phosphoric acid ester structure, and a silyl group. These substituents may be substituted with any of these substituents.

Further, the aryl group represented by Q in General Formula (2) is either a monocyclic or a condensed ring aryl group which has preferably 6-24 carbon atoms and more preferably 6-20 carbon atoms. Listed as such monocyclic or condensed ring aryl groups may be, for example, a phenyl group, a naphthyl group, an anthracenyl group, a naphthacenyl group, and a triphenylenyl group. Incidentally, the aryl group represented by Q may have a substituent which does not adversely affect the formation of images. Listed as such substituents are a halogen atom, an alkyl group, an alkenyl group, an alkynyl group, an aryl group, a heterocyclic group (including an N-substituted nitrogen containing heterocyclic group such as a morpholino group), an alkoxycarbonyl group, an aryloxycarbonyl group, a carbazoyl group, a cyano group, a thiocarbamoyl group, an aryloxy group, a heterocyclicoxy group, an acyloxy group, an (alkoxy or aryloxy)carbonyloxy group, a sulfonyloxy group, an acylamide group, a sulfonamide group, a ureido group, a thioureido group, an imido group, an (alkoxy or aryloxy)carbonylamino group, a sulfamoyl group, a semicarbazide group, a thiosemicarbazido group, an (alkyl or aryl)sulfonylureido group, a nitro group, an (alkyl or aryl)sulfonyl group, a sulfamoyl group, a group comprised of a phosphoric amide or phosphoric acid ester structure, and a silyl group. These substituents may be substituted with any of these substituents.

Further, the heterocyclic groups represented by Q in General Formula (2) is a 4- to 8-membered saturated or unsaturated heterocyclic group containing at least one of the atoms consisting of nitrogen, oxygen, sulfur, selenium, and tellurium. These groups may be monocyclic or may form a condensed ring with another ring. Such a heterocyclic group is preferably a 5- to 6-membered unsaturated heterocyclic ring which may have a condensed ring. Preferably listed as heterocycles in the heterocyclic group which may have such a condensed ring may be imidazole, pyrazole, pyridine, pyrimidine, pyrazine, pyridazine, triazole, triazine, indole, indazole, purine, thiadiazole, oxadiazole, quinoline, phthalazine, naphthyridine, quinoxaline, quinazoline, cinnoline, pteridine, acridine, phenanthroline, phenazine, tetrazole, thiazole, oxazole, benzimidazole, benzoxazole, benzothiazole, indolenine, and tetraazaindene. More preferably listed may be imidazole, pyridine, pyrimidine, pyrazine, pyridazine, triazole, triazine, thiadiazole, oxadiazole, quinoline, phthalazine, naphthyridine, quinoxaline, quinazoline, cinnoline, tetrazole, thiazole, oxazole, benzimidazole, benzoxazole, benzothiazole, and tetraazaindene. Incidentally, the heterocyclic group represented by Q may have any substituent which does not adversely affect the formation of images. Listed such substituents may be the same substituents as those of the aforesaid aryl group.

Listed as halogen compounds represented by General Formula (2) may be, for example, the compounds illustrated below.

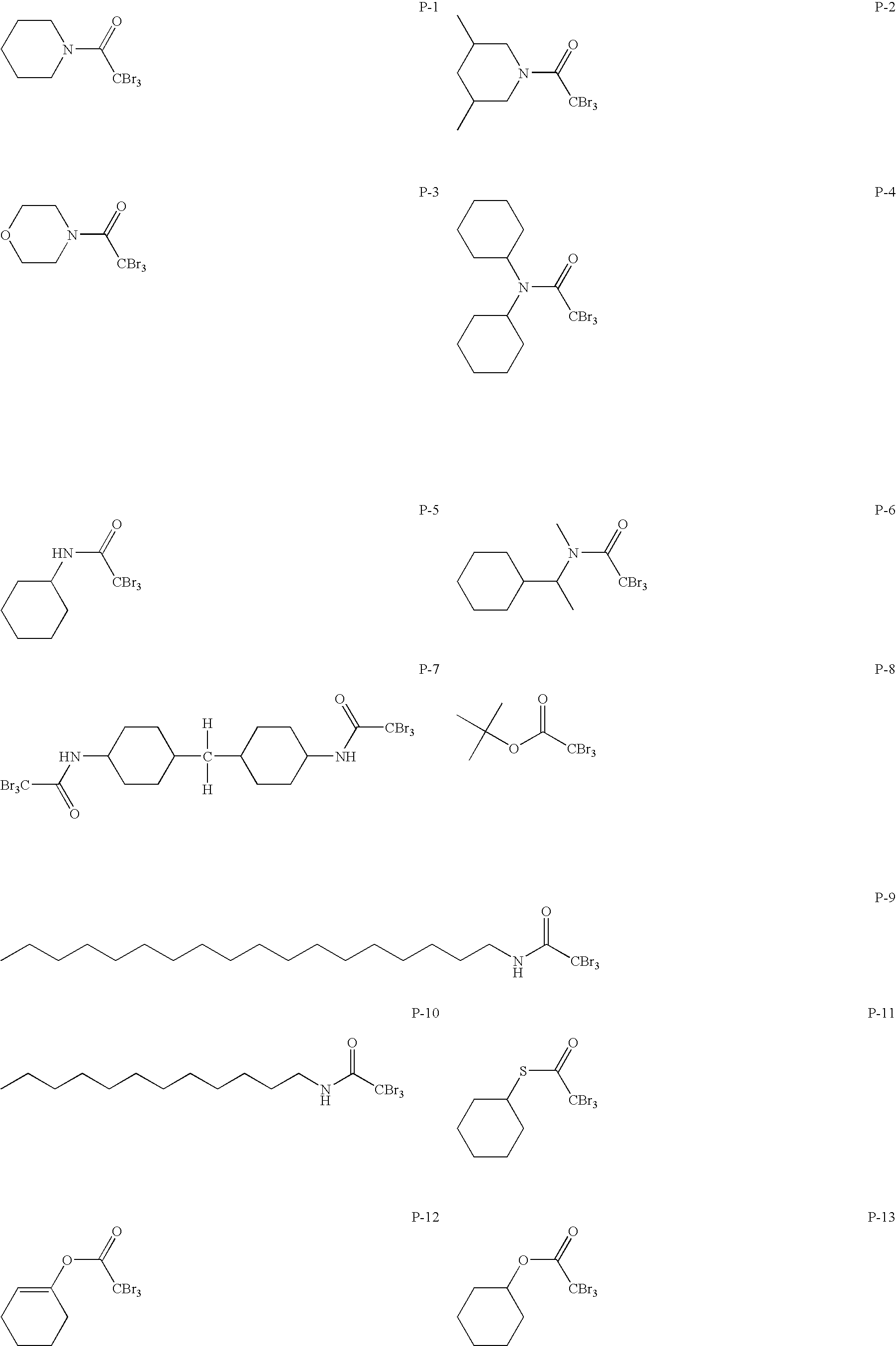

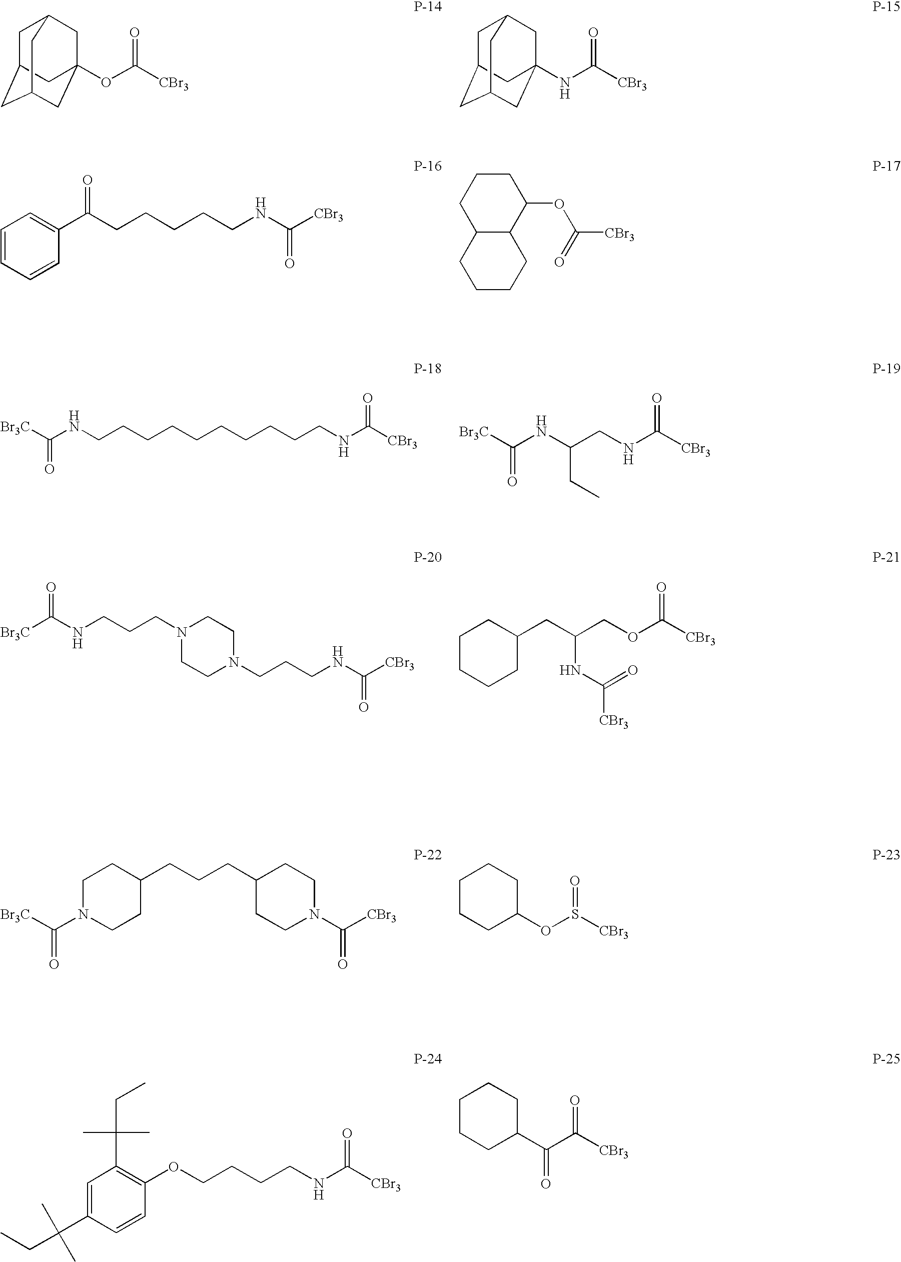

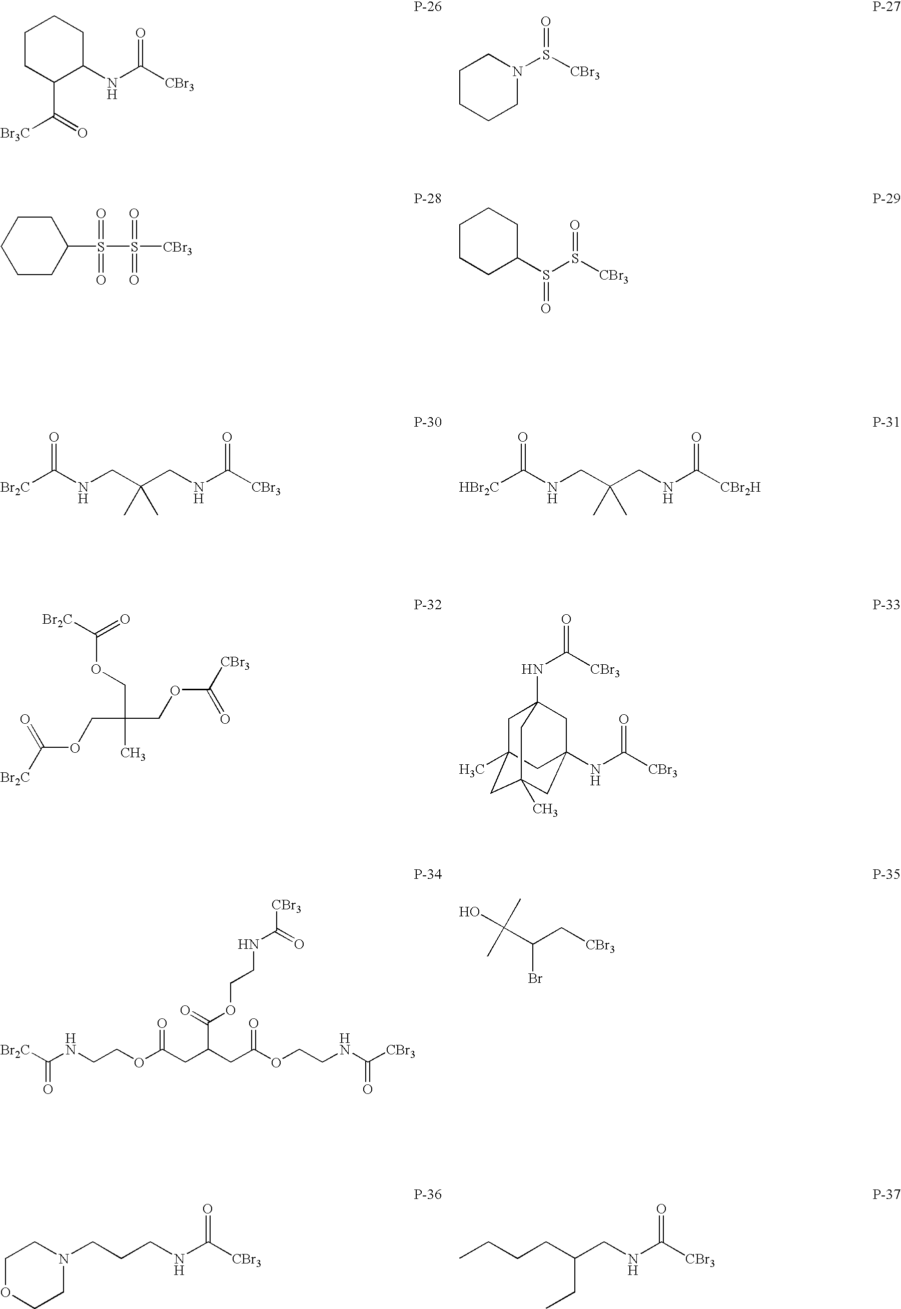

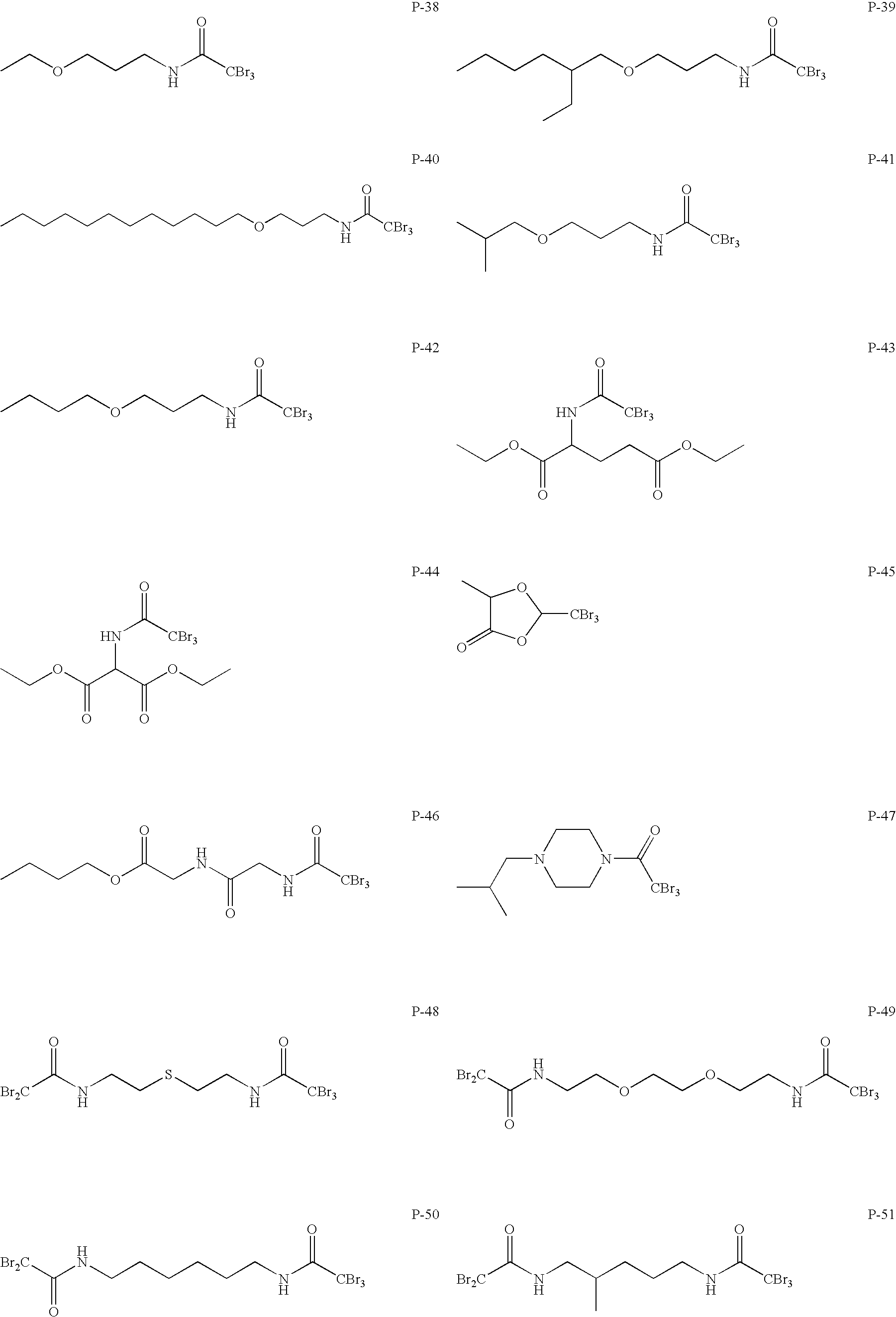

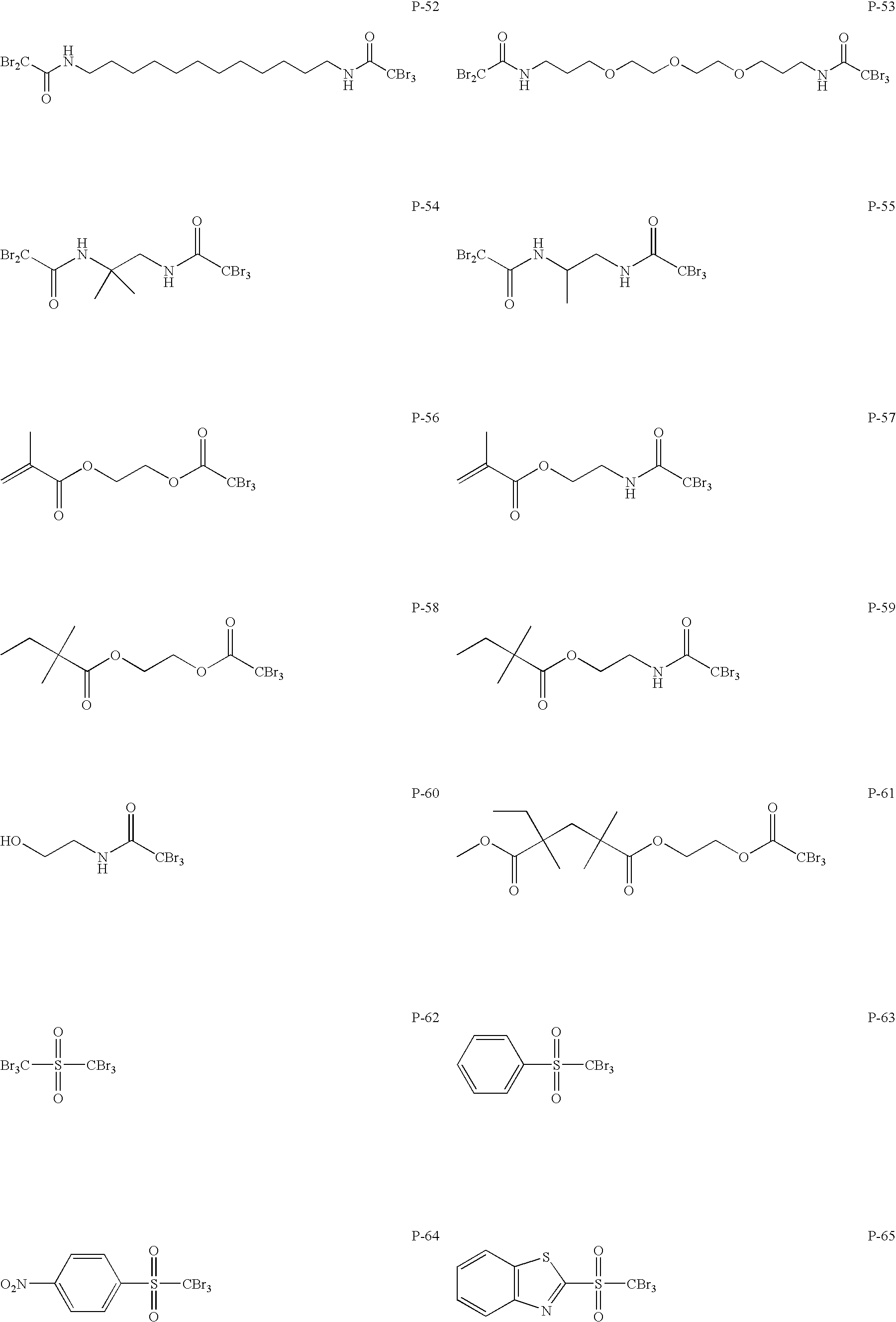

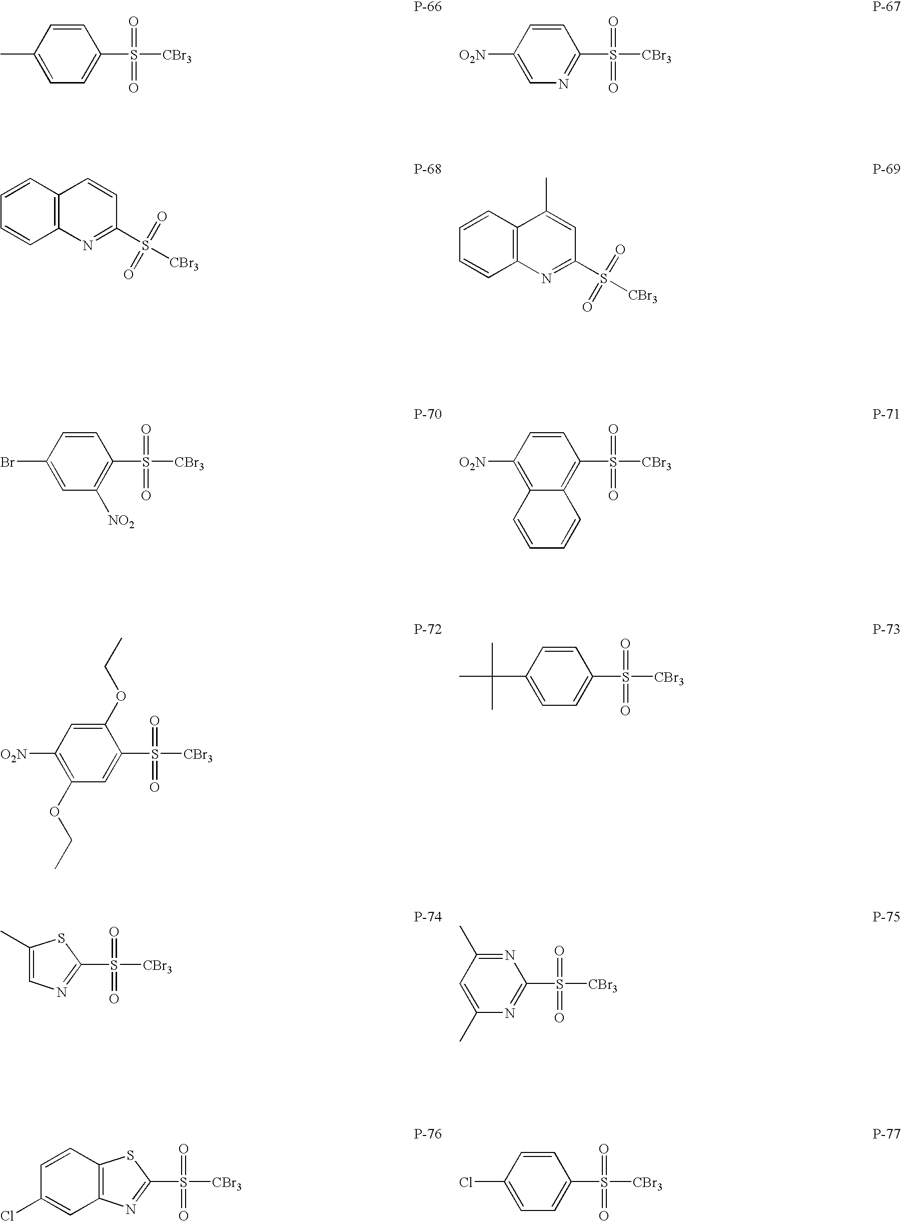

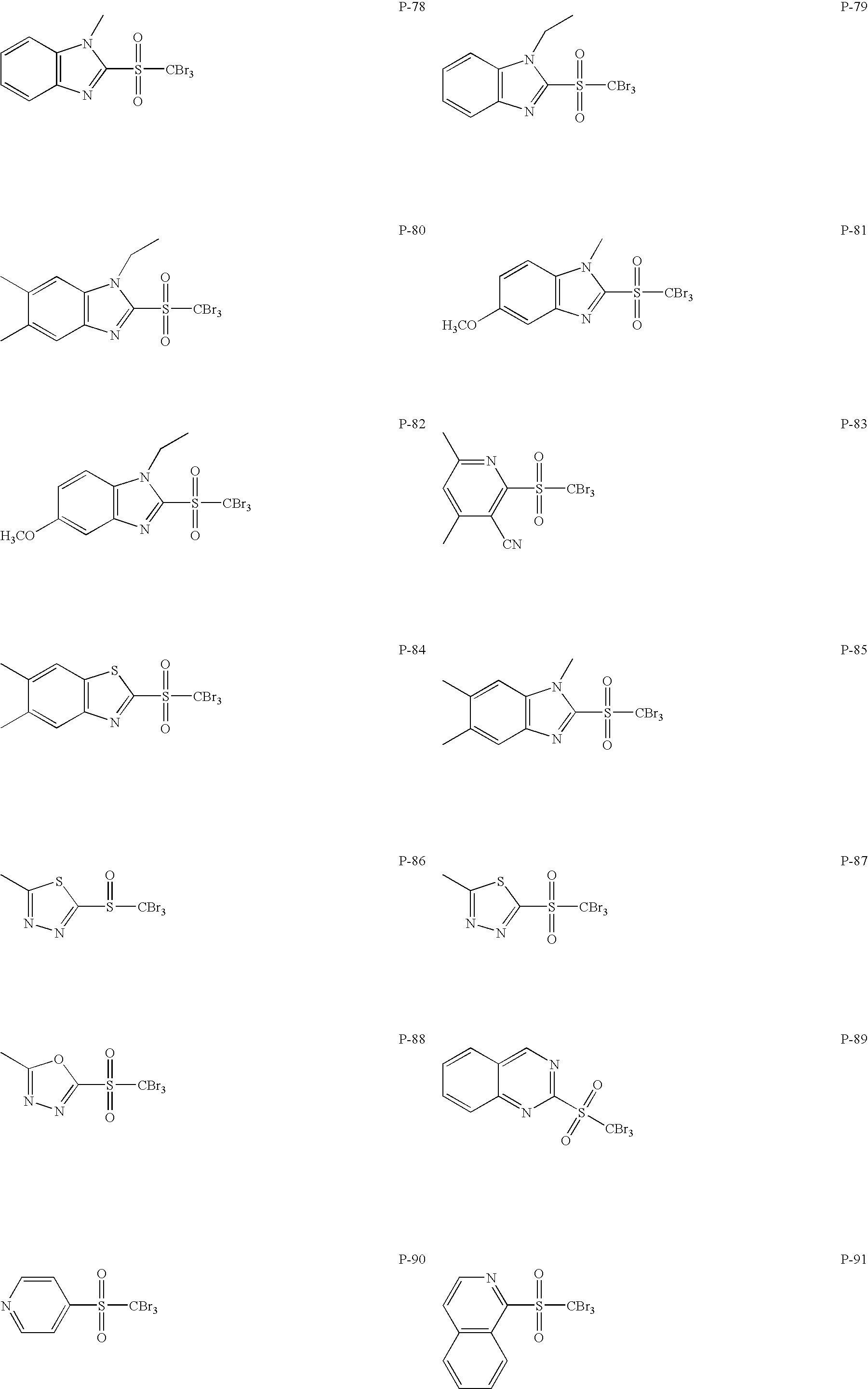

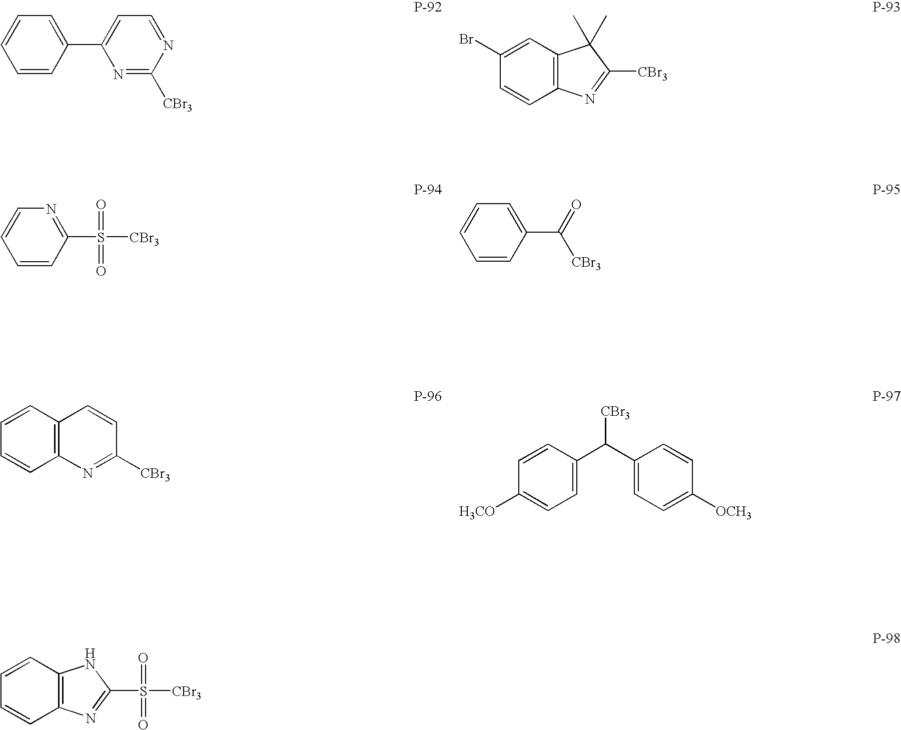

Further, if the objectives of the present invention are not adversely affected, it is possible to suitably select and use compounds disclosed in U.S. Pat. Nos. 3,874,964, 4,756,999, 5,028,523, 5,340,712, 5,369,000, and 5,464,737; European Patent Nos. 600,587, 605,981, and 631,176; Japanese Patent Publication Nos. 54-44212, 51-9694, 50-137126, 50-89020, 50-119624, 55-140833, and 59-57234; and JP-A Nos. 7-2781, 7-5621, 9-90550, 9-160164, 9-160167, 9-244177, 9-244178, 9-258367, 9-265150, 9-288328, 9-319022, 10-197988, 10-197989, 11-242304, 2000-2963, 2000-112070, 2000-284412, 2000-284410, and 2001-33911. These compounds may be employed individually or in combinations of at least two types.

In the range in which the objectives of the present invention are not adversely affected, added, in addition to the aforesaid halogen compounds, as antifoggants may be polycarboxylic acids or anhydrides thereof described in JP-A Nos. 58-107534, 8-6203, 2000-1999936, 2000-321711, and 2002-23304, 2002-49121, thiosulfonic acid or salts thereof, or derivatives thereof described in JP-A Nos. 51-78227, 53-20923, 55-140833, 7-209797, 8-314059, 9-43760, 2000-284400, and 2000-284413, carboxylic acid and sulfonic acid or salts thereof described in U.S. Pat. No. 6,083,681, and JP-A Nos. 2002-62616, 200262617, and 2002-90935, and vinyl based compounds described in U.S. Pat. No. 5,686,228.

Examples of toning agents, which are added to improve silver tone after development, include imides (e.g., phthalimide); cyclic imides, pyrazoline-5-ones and quinazolinones (e.g., succinimide, 3-phenyl-2-pyrazoline-5-one, 1-phenylurazole, quinazoline, and 2,4-thiazolidinone); naphthalimides (e.g., N-hydroxy-1,8-naphthalimide); cobalt complexes (e.g., cobalt hexaaminetrifluoroacetate), mercaptans (e.g., 3-mercapto-1,2,4-triazole); N-(aminomethyl)aryldicarboxyimides (e.g., N-(dimethylaminomethyl)phthalimide); blocked pyrazoles, isothiuronium derivatives and combinations of a certain type of light-bleaching agents (e.g., a combination of N,N′-hexamethylene(1-carbamoyl-3,5-dimethylpyrazole, 1,8-(3,6-dioxaoctane)bis(isothiuroniumtrifluoroacetate), and 2-(tribromomethylsulfonyl)benzothiazole); merocyanine dyes (e.g., 3-ethyl-S-(3-ethyl-2-benzothiazolinidene(benzothiazolinylidene))-1-methylethylethylidene)-2-thio-2,4-oxazolidinedione); phthalazinone, phthalazinone derivatives, or metal salts of derivatives thereof (e.g., 4-(1-naphthyl)phthalazinone, 6-chlorophthalazinone, 5,7-dimethyloxyphthalazinone, and 2,3-dihyro-1,4-phthalazinedione; combinations of phthalazinone and sulfinic acid derivatives (e.g., 6-chlorophthalazinone and sodium benzenesulfinate or 8-methylphthaladinone and sodium p-trisulfonate); combinations of phthalazine and phthalic acid; combinations of phthalazine (including phthalazine addition products) with at least one compound selected from the group consisting of maleic anhydride, and phthalic acid, 2,3-naphthalenedicarboxylic acid or o-phenylenic acid and anhydride thereof (e.g., phthalic anhydride, 4-methylphthalic anhydride, 4-nitrophthalic anhydride, and tetrachlorophthalic anhydride); quinazolinediones, benzoxazine, naphthoxazine derivatives; benzoxazine-2,4-diones (e.g., 1,3-benzoxazine-2,4-dione); pyrimidines and asymmetry-triazines (e.g., 2,4-dihydroxypyrimidine), and tetraazapentalene derivatives (e.g., 3,6-dimercapto-1,4-diphenyl-1H,4H-2,3a,5,6-tetraazapentalene). Listed as preferred toning agents are combinations of phthalazone or derivatives thereof, phthalazine or derivatives thereof, or phthalazinone or derivatives thereof with phthalic acid or derivatives thereof. Of these, the combinations of phthalazine or derivatives thereof along with phthalic acid or derivatives thereof are particularly preferred.

Further, toning agents may be incorporated into the protective layer, barrier layer, and interlayer, which are described below.

Still further, it is possible to use sensitizing dyes without any particular limitation, as long as they absorb wavelengths of a laser beam transmitted from a laser beam source which is employed for the scanning exposure, to be detailed later in the image forming method.

Of these, from the viewpoint of maintenance as well as the size of light sources, preferred as laser beam sources are semiconductor laser beam sources with a wavelength of 700-1,200 nm. Employed as sensitizing dyes which exhibit the maximum absorption wavelength in the wavelength region above may be, for example, cyanine dyes, rhodacyanine dyes, oxonol dyes, carbocyanine dyes, dicarbocyanine dyes, tricarbocyanine dyes, tetracarbocyanine dyes, pentacarbocyanine dyes, styryl dyes, pyrylium dyes, metal phthalocyanine dyes, and metal containing dyes such as metal porphyrin. Specifically, it is possible to select and use the dyes which are described in Chem. Rev., 92, 1197 (1992), and JP-A Nos. 48-3527, 49-11121, 58-145936, 59-191032, 59-1922242, 60-80941, 62-284343, 2-105135, 3-67242, 3-163440, 4-182639, 5-341432, 7-13295, 11-30833, 11-352628, 2000-95958, 2000-98524, 2000-122207, 2000-169741, 2000-171938, 2000-273329, 2000-321704, 2001-64527, and 2001-83655, and Japanese Patent Publication Open to Public Inspection (under PCT Application) No. 9-510022.

Further, employed as supersensitizers may be suitably selected compounds which are described in RD Item 17643, Japanese Patent Publication No. 9-25500 and 43-4933, and JP-A Nos. 59-19032, 59-1922542, and 5-341432. In the present invention, it is possible to employ hetero-aromatic mercapto compounds represented by General Formula (7), below, and basically disulfide compounds represented by General Formula (8) which form the aforesaid mercapto compounds.

Ar—SM General Formula (7)

Ar—S—S—Ar General Formula (8)

In General Formula (7), M represents a hydrogen atom or an alkaline metal atom, while Ar represents an aromatic heterocyclic ring or a condensed aromatic heterocyclic ring, having at least one of the nitrogen, sulfur, oxygen, selenium or tellurium atom. Aromatic heterocyclic rings are preferably benzimidazole, naphthoimidazole, benzothiazole, naphthothiazole, benzoxazole, naphthoxazole, benzoselenazole, benzotetrazole, imidazole, oxazole, pyrazole, triazole, triazine, pyrimidine, pyridazine, pyrazine, purine, quinoline, or quinazoline. Further, Ar in General Formula (8) is as defined in aforesaid General Formula (7).

The aforesaid aromatic heterocyclic ring may have substituent(s) selected from the group consisting of, for example, a halogen atom (such as Cl, Br, or I), a hydroxy group, an amino group, a carboxyl group, an alkyl group (for example, having at least one carbon atom or having preferably 1-4 carbon atoms), or an alkoxy group (for example, having at least one carbon atom, or having preferably 1-4 carbon atoms).

Further, in the image forming material employed in the present invention, it is preferable that thiuronium compounds, described in JP-A No. 2001-330918, is selected as a supersensitizer for the purpose of achieving high sensitivity. Further, such supersensitizers are incorporated into the image forming layer comprising photosensitive silver halide as well as non-photosensitive organic silver salts in an amount ranging preferably from 0.0001 to 1.0 mol per mol of silver, and more preferably from 0.001 to 0.5 mol.

Further, it is possible to incorporate macrocyclic compounds containing hetero atom(s) into the image forming layer of the present invention. Preferred as such macrocyclic compounds are those comprising of at least 9-membered rings containing, as a hetero atom, at least a nitrogen atom, an oxygen atom, a sulfur atom, and a selenium atom, and 12- to 24-membered rings are more preferred. In 1967, synthesized as such compounds was crown ether by C. J. Pederson, as listed below. After publication of his dramatic report, many macrocyclic compounds were synthesized. Macrocyclic compounds are detailed in C. J. Pederson, Journal of American Chemical Society, Vol. 86 (2495), 7017-7036 (1967), G. W. Gokel, S. H. Korzeniowski, “Macrocyclic Polyether Synthesis”, Springer-Vergal, (1982), and Japanese Patent Application Open to Public Inspection No. 2000-347343.

Further, other than the aforesaid additives, employed may be, for example, surfactants, antioxidants, stabilizers, plasticizers, UV absorbers, and covering aids. It is possible to select and use these compounds and compounds described in RD Item 17029 (June 1979, pages 9-15) in any range in which the purposes of the present invention are not adversely affected.

The image forming layer of the present invention may be comprised of a single layer or a plurality of layers having the same or different compositions. The thickness of the image forming layer is customarily 5-30 μm.

Further, the image forming material employed in the present invention comprises the aforesaid support coated thereon an image forming layer and a protective layer in the stated order. The protective layer is comprised of binder resins described in the aforesaid backing layer and/or image forming layer and suitably selected additives added as required.

For the purpose of reducing image abrasion after thermal development as well as assuring desired tracking properties, it is preferable that fillers are incorporated into the protective layer as an additive. When the aforesaid fillers are added, those fillers are usually incorporated into layer forming compositions in an amount of 0.05-30 percent by weight.

Further, in order to improve lubricating property and electrification property, suitably selected lubricants and antistatic agents may be incorporated into the protective layer. Suitably selected and employed as such compounds may be lubricants and antistatic agents employed in the backing layer.

Still further, when the binder resins of the protective layer comprises a hydroxyl group or active hydrogen, layer strength may be enhanced by adding crosslinking agents such as a metal alkoxide which comprises, in the molecule, a plurality of metal alkoxide portions of conventional multifunctional isocyanate compounds, alkoxysilane compounds, and alkoxytitanium compounds, all known in the art.

The added amount of these additives is preferably about 0.01-about 20 percent by weight with respect to the protective layer forming components, and is more preferably 0.05-10 percent by weight.

The protective layer of the image forming material employed in the present invention may be comprised of a single layer or a plurality of layers. Incidentally, the thickness of the protective layer is customarily 1.0-5.0 μm.

In the image forming material employed in the present invention, other than the image forming layer and the protective layer, other layers may be provided on one surface of the aforesaid support. For example, for the purpose of adjusting adhesion force, an interlayer may be provided between the support and the image forming layer. Further, a barrier layer may be provided between the image forming layer and the protective layer, for example, to minimize the transfer of compounds, which tend to float up from the image forming layer to the surface, and oxygen and moisture which reach the image forming layer through the protective layer, and to achieve sufficient adhesion force between the image forming layer and the protective layer.

The aforesaid interlayer and barrier layer are comprised of binder resins described in the aforesaid backing layer and/or the image forming layer as well as any desired additives. These may be suitably selected and used. Each of these layers may be comprised of a single layer or a plurality of layers having the same or different compositions. Incidentally, the thickness of these layers is customarily 0.01-5.0 μm.

Further, liquid coating compositions of the image forming layer as well as the protective layer which are provided in the image forming material employed in the present invention and any desired backing layer, interlayer, and barrier layer which are provided, if desired, are prepared by dissolving or dispersing the aforesaid components in or into respective solvents.

From the viewpoint of solubility of resins as well as drying properties during production, it is preferable to employ solvents comprised of carbon, hydrogen, and oxygen atoms, which exhibit a solubility parameter value in the range of 15.0-30.0, which is shown in “Polymer Handbook, Fourth Edition”, 675 (John Wiley & Sons, Inc. 1998).

Listed as such solvents are, for example, ketones such as acetone (20.3), isoforon (18.6), ethyl amyl ketone (16.8), methyl ethyl ketone (19.0), methyl isobutyl ketone (17.2), cyclopentanone (21.3), or cyclohexanone (20.3); alcohols such as methyl alcohol (29.7), ethyl alcohol (26.0), n-propyl alcohol (24.3), isopropyl alcohol (23.5), n-butyl alcohol (23.3), isobutyl alcohol (21.5), t-butyl alcohol (21.7), 2-butyl alcohol (22.1), diacetone alcohol (18.8), or cyclohexanol (23.3); glycols such as ethylene glycol (29.9), diethylene glycol (34.8), triethylene glycol (21.9), or propylene glycol (25.8); ether alcohols such as ethylene glycol monomethyl ether (23.3); ethers such as diethyl ether (15.1), tetrahydrofuran (18.6), 1,3-dioxysolan (17.6), or 1,4-dioxane (16.2); esters such as ethyl acetate (18.6), n-butyl acetate (17.4), or 2-butyl acetate (16.8); and hydrocarbons such as n-heptane (15.1), cyclohexane (16.8), toluene (18.2), or xylene (18.0). Solvents are not limited to those listed above as long as the solubility parameter value (numerical figure in parentheses) is in the aforesaid range. Further, these solvents may be employed individually or in combinations of several types.

During preparation of liquid coating compositions, when dispersion is required, suitably selected and employed may be conventional homogenizers known in the art, such as a two-roller mill, a three-roller mill, a ball mill, a pebble mill, a Cobol mill, a tron mill, a sand mill, a sand grinder, a Sqegvari attritor, a high speed impeller homogenizer, a high speed stone mill, a high speed impact mill, a disper, a high speed mixer, a homogenizer, an ultrasonic homogenizer, an oven kneader, and a continuous kneader.

It is possible to apply the liquid coating composition prepared as above onto a support, employing any coater selected from, for example, an extrusion system extrusion coater, a reverse roll coater, a gravure roll coater, an air doctor coater, a blade coater, an air knife coater, a squeeze coater, an impregnated coater, a bar coater, a transfer roll coater, a kiss coater, a casting coater, a spray coater, and a slide coater which are known in the art. Of these coaters, in order to minimize fluctuation of the thickness of the aforesaid layer, it is preferable to employ roll coaters such as an extrusion system extrusion coater as well as a reverse roll coater.

Further, in the case of coating the protective layer, coaters are not particularly limited as long as the image forming layer is not damaged. However, when solvents employed in the protective layer forming liquid coating composition exhibit the possibility of dissolving the aforesaid image forming layer, of the aforesaid coaters, it is possible to employ an extrusion system extrusion coater, a gravure roll coater or a bar coater. Incidentally, of these, when a contact coating method in which the gravure roll coater or the bar coater is employed, the rotation direction of the gravure roll or the bar may be in either direction. In the case of the normal rotation, the rotation rate may be the same or may result in a difference in peripheral rotation rate.

Further, coating and drying may be repeated for each layer. However, multilayer coating may be carried out employing a wet-on-wet system and subsequently, drying may be performed. In such a case, it is possible to carry out coating in combinations of the aforesaid reverse roll coater, gravure roll coater, air doctor coater, blade coater, air knife coater, squeeze coater, impregnated coater, bar coater, transfer roll coater, kiss coater, casting coater, spray coater, slide coater, and extrusion system extrusion coater. In multilayer coating under the wet-on-wet system, the upper layer is applied onto the lower layer while it is still wet, whereby adhesion between the upper layer and the lower layer is enhanced. In this case, when a multilayer is comprised of at least two layers, no particular limitation is imposed.

Further, when an interlayer forming liquid coating composition or an image forming layer liquid coating composition is applied onto a support, it is preferable that the surface of the support is previously subjected to at least one of the following surface treatments: a flame treatment, an ozone treatment, a glow discharge treatment, a corona discharge treatment, a plasma treatment, an ultraviolet radiation exposure vacuum reatment, and a radiation exposure treatment. By applying such a surface treatment to the surface of the support, it is possible to ensure the adhesion between the support and the image forming layer.

In the invention, “plural image forming materials being different from each other” means that the plural image forming materials are different in their characteristic. For example, “a first image forming material being different from a second image forming material” means the first image forming material has at least one characteristic being different from that of the second image forming material. However, in the invention, when the plural image forming material are different in only their size, those image forming materials are not regarded as being different from each other.

Especially, in the invention, it is preferable that the plural image forming materials are different in a color tone or a maximum density of their image, when the images are formed in the same condition on the plural image forming materials. In the invention, it is also preferable that the plural image forming materials are different in at least one of their sensitivity, transmittance and gradient. Further, it is also preferable that one of the plural image forming material has a transparent support and another has a reflective support.

One example of the image forming materials, which are different from each other, employed in the image forming apparatus, image forming method and image forming system of the present invention will now be shown. However, image forming materials employed in the invention are not limited to the materials, described below.

### IMAGE FORMING MATERIAL 1

### Image Forming Material Comprised of a Blue Tinted Transparent Support

<Preparation of Backing Layer Forming Liquid Coating Composition>

A backing layer forming liquid coating composition was prepared employing the method described below.

While stirring, added to 83.0 g of methyl ethyl ketone were 8.42 g of cellulose acetate propionate (CAP482-20, manufactured by Eastman Chemical Co.) and 0.45 g of polyester resin (Vylon 280, manufactured by TOYOBO Co., Ltd.). After dissolution, 1.03 g of Infrared Dye 1 was added.

Separately, dissolved in 4.32 g of methanol were 0.64 g of a fluorine based surfactant (Surflon S-381 (effective component of 70 percent), manufactured by Asahi Glass Co., Ltd.) and 0.23 g of a fluorine based surfactant (Megafag F120K, manufactured by Dainippon Ink and Chemicals, Inc.). Subsequently, added to a solution containing Infrared Dye 1, described below, was a fluorine based surfactant solution and the resulting mixture was stirred well until Infrared Dye 1 was completely dissolved. Finally, 7.5 g of silica (Sylophobic 4004, manufactured by Fuji Silysia Chemical Ltd.), which was dispersed in methyl ethyl ketone at a concentration of one percent employing a dissolver type homogenizer) and 1.78 g of an isocyanate compound (Coronate C-3041, manufactured by Nippon Polyurethane Industry Co., Ltd.) which was diluted with methyl ethyl ketone to result in a solid concentration of 20 percent by weight were successively added while stirring, whereby a backing layer forming liquid coating composition was prepared.

<Coating of Backing Layer>Subsequently, employed as a support was a 175 μm thick biaxially oriented polyethylene terephthalate film tinted with a blue dye (Ceres Blue RR-J, manufactured by Bayer A G) to result in a visual transmission density of 0.157 (measured by PDA-65, manufactured by Konica Corp. to three places of decimals). One surface was then subjected to a plasma treatment under a high frequency output of 4.5 kW, a frequency of 5 kHz, a treatment time of 5 seconds and gas conditions at a volume ratio of argon, nitrogen, and hydrogen of 90, 5, and 5 percent respectively, employing a batch type atmospheric plasma treatment apparatus (AP-I-H-340, manufactured by E. C. Kagaku Co., Ltd.). Subsequently, the opposite surface was subjected to a corona discharge treatment (at 40 W/m² minute) and the aforesaid backing layer forming liquid coating composition was applied onto the corona discharged surface to result in a dried layer thickness of 3.50 μm, employing an extrusion coater and subsequently dried, whereby a backing layer was formed.

<Preparation of Image Forming Layer Forming Liquid Coating Composition 1>

<Preparation of Photosensitive Silver Halide Emulsion 1>

Dissolved in 900 ml of water were 7.5 g of ossein gelatin of an average molecular weight of 100,000 and 10 mg of potassium bromide, and the pH of the resulting solution was adjusted to 3.0 at 35° C. Thereafter, 370 ml of an aqueous solution containing 74 g of silver nitrate and 370 ml of an aqueous solution containing potassium bromide and potassium iodide at a mol ratio of 98/2 in the same mol as silver nitrate, and iridium chloride in an amount of 1×10⁻⁴ mol per mol of silver were added employing a controlled double jet method over a span of 10 minutes, while maintaining the pAg at 7.7. Subsequently, 0.3 g of 4-hydroxy-6-methyl-1,3,3a,7-tatraazaindene was added, and the pH was adjusted to 5 by adding NaOH, whereby cubic silver iodobromide grains were prepared which had an average grain size of 0.06 μm, a grain size variation coefficient of 12 percent, and a [100] plane ratio of 87 percent.

The resulting emulsion was coagulated employing gelatin coagulants and then desalted. Thereafter, 0.1 g of phenoxyethanol was added and the pH and pAg were adjusted to 5.9 and 7.5, respectively, whereby Photosensitive Silver Halide Emulsion 1 was prepared.

(Preparation of Photosensitive Organic Silver Salt A)

At 80° C., dissolved in 4,720 ml of pure water were 171.2 g of behenic acid, 49.4 g of arachidic acid, and 34.4 g of stearic acid. Subsequently, while stirring at high speed, 540.2 ml of a 1.5 mol/L aqueous sodium hydroxide solution was added, and 6.9 ml of concentrated nitric acid was then added. Thereafter, the resulting mixture was cooled to 55° C., whereby a fatty acid sodium salt solution was prepared.

While maintaining the temperature of the aforesaid fatty acid sodium salt solution at 55° C., Photosensitive Silver Halide Emulsion 1 (containing 0.038 mol of silver), prepared as above, and 450 ml of pure water were added and stirred for 5 minutes. Subsequently, 760.6 ml of a 1 mol/L silver nitrate solution was added over a span of two minutes and stirred for an additional 20 minutes. Subsequently, water-soluble salts were removed by filtration. Thereafter, washing and filtration were repeated employing deionized water until the conductivity of the filtrate reached 2 μS/cm and then centrifugal dehydration was carried out. Thereafter, until the weight no longer deceased at 37° C., drying was carried out employing heated air, whereby Powdered Photosensitive Organic Silver Salt A was prepared.

(Preparation of a Photosensitive Emulsion)

Dissolved in 291.4 g of methyl ethyl ketone was 2.91 g of polyvinyl butyral resin (S-lec BL-5Z at a hydroxyl group valence of 175, manufactured by Sekisui Chemical Co., Ltd.). While stirring employing a dissolver type homogenizer, 100 g of Photosensitive Organic Silver Salt A, prepared as above, was gradually added and mixed well. Thereafter, dispersion was performed at a peripheral rate of 13 m and a retention time of 0.5 minute in the mill, employing a media homogenizer (manufactured by Gettzmann Co.) filled with zirconia beads of a particle diameter of 0.5 mm at 80 percent of capacity, whereby a photosensitive emulsion was prepared.

While stirring, 50 g of the aforesaid photosensitive emulsion and 10.0 g of methyl ethyl ketone were mixed while maintained at 18° C. Subsequently, 0.312 g of Antifoggant 1 methanol solution (11.2 percent) was added and stirred for one hour. Further, 0.418 g of a calcium bromide methanol solution (11.2 percent) was added and stirred for 20 minutes. Subsequently, 0.337 g of a solution, which was separately prepared by dissolving 0.894 g of dibenzo-18-crown-6 and 0.279 g of potassium acetate in 10.0 g of methanol, was added and stirred for 10 minutes. Subsequently, 4.753 g of the dye solution described below was added and stirred for 60 minutes. Thereafter, the temperature was lowered to 13° C. and stirring was carried out for an additional 50 minutes.

<Dye Solution>

| | |
|---|---|
| Infrared Sensitizing Dye 1 | 0.0148 g |
| (2-Carboxyphenyl)-4-methylbenzenesulfonate | 6.372 g |
| 2-Chlorobenzoic acid | 0.739 g |
| Methyl ethyl ketone | 40.00 g |

While maintaining the dye solution at 13° C., 0.399 g of a Thiuronium Compound 1 methanol solution (0.94 percent) was added and stirred for 5 minutes. Thereafter, 15.32 g of polyvinyl butyral (S-lec BL-5Z having a hydroxyl group valence of 175, manufactured by Sekisui Chemical Co., Ltd.) and stirred for 10 minutes. Subsequently, 0.180 g of tetrachlorophthalic acid was added and dissolved while stirring for 30 minutes, whereby Solution A was prepared.

Image Forming Layer Forming Liquid Coating Composition 1 was prepared by successively adding while stirring, to the resulting Solution A, each of Additive Solutions 1, 2, 3, 4, and 5 in an amount of 0.974 g, 2.989 g, 13.543 g, 3.570 g, and 6.461 g, respectively.

(Additive Solution 1)

A solution prepared by dissolving an isocyanate compound (Coronate HX, manufactured by Nippon Polyurethane Industry Co., Ltd.) in methyl ethyl ketone at a solid concentration of 50.0 percent)

(Additive Solution 2)

Solution prepared by dissolving potassium p-toluenesufonate in methyl ethyl ketone at a solid concentration of 20 percent

<Additive Solution 3>

| | |
|---|---|
| 1,1-Bis(2-hydroxy-3,5-dimethylphenyl)- | 10.57 g |
| (2,4-dimethyl-3-cyclohexenyl)methane | |
| 4-Methylphthalic acid | 0.588 g |
| Infrared Dye 1 | 0.0354 g |
| Methyl ethyl ketone | 50.00 g |

<Additive Solution 4>

A solution prepared by dissolving a trihalomethyl group containing compound (Exemplified Compound P-30) at a solid concentration of 10.85 percent.

<Additive Solution 5>

A solution prepared by dissolving phthalazine in methyl ethyl ketone at a solid concentration of 6.63 percent.

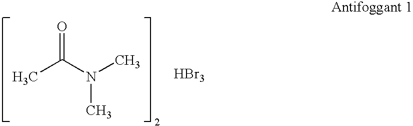

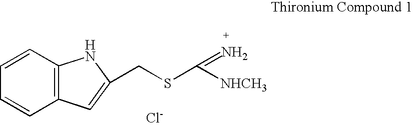

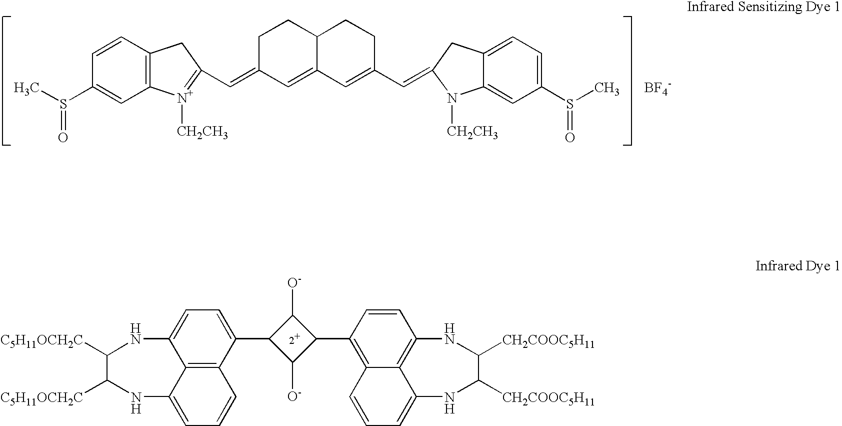

<Preparation of a Protective Layer Forming Liquid Coating Composition>

While stirring, added to and dissolved in 40.0 g of methyl ethyl ketone were 10.05 g of phenoxy resin (PKHH, manufactured by InChem Corp.), 0.013 g of benzotriazole, and 0.10 g of fluorine based surfactant (Surfron KH40, manufactured by Asahi Glass Co., Ltd.). Subsequently, 2.00 g of a polyisocyanate compound at 50 percent solid (Coronate 3041, manufactured by Nippon Polyurethane Industry Co., Ltd.) was added while stirring, whereby a protective layer resinous solution was prepared.

Separately, a silica dispersion was prepared by adding 5.0 g of hydrophobic silica (Sylophobic 200, manufactured by Fuji Silysia Chemical Ltd.) to 55.0 g of methyl ethyl ketone and subsequently dispersing the resulting mixture employing a ultrasonic homogenizer. Thereafter, while stirring the aforesaid protective layer resinous solution, 3.0 g of silica dispersion was added and the resulting mixture was subjected to ultrasonic dispersion, whereby a protective layer forming liquid coating composition was prepared.

(Coating the Image Forming Layer Side)

Image Forming Layer Forming Liquid Coating Composition 1 and the protective layer forming liquid coating composition, which were prepared employing the aforesaid method, were subjected to multilayer coating onto a plasma treated surface of the support provided with the aforesaid backing layer, while employing an extrusion coater, and subsequently dried by 75° C. air flow, whereby Image Forming Material 1 was prepared. The thickness of the image forming layer was controlled to result in a silver amount of 1.85±0.05 g/m², and the protective layer was controlled to an amount of 2.00±0.05 g/m².

### IMAGE FORMING MATERIAL 2

### Image Forming Material which Results in Different Tone Compared to Image Forming Material 1

<Preparation of Image Forming Layer Forming Liquid Coating Composition 2>

Additive Solution 6 was prepared by adding 0.529 g of 1,1-bis(4-hydroxy-3,5-di-t-butylphenyl)methane to Additive Solution 3 employed in Image Forming layer Forming Liquid Coating Composition 1. Subsequently, while stirring, Additive Solutions 1, 2, 6, 4, and 5 were successively added to Solution A prepared for Image Forming Layer Forming Liquid Coating Composition 1 in an amount of 0.974 g, 2.989 g, 13.660 g, 3.570 g, and 6.461 g, respectively, whereby Image Forming Layer Forming Liquid Coating Composition 2 was prepared.

<Coating the Image Forming Layer Side)

Image Forming Layer Forming Liquid Coating Composition 2, prepared employing the aforesaid method and the same protective layer forming liquid coating composition as Image Forming Material 1, was subjected to multilayer coating onto a plasma treated surface of the support provided with the same backing layer as Image Forming Material 1, while employing an extrusion coater, and subsequently dried by 75° C. air flow, whereby Image Forming Material 2 was prepared. The thickness of the image forming layer was controlled to result in a silver amount of 1.85±0.05 g/m², and the protective layer was controlled to an amount of 2.00±0.05 g/m².

### IMAGE FORMING MATERIAL 3

### Image Forming Material which Results in Different Maximum Density Compared to Image Forming Material 1

<Preparation of Image Forming Layer Forming Liquid Coating Composition 3>

Additive Solution 7 was separately prepared by dissolving N-(2-aminoethyl)-3-aminopropyltrimethoxysilane in methanol to result in a solid concentration of 20 percent. Subsequently, while stirring, Additive Solutions 1, 2, 3, 4, 5, and 7 were successively added to Solution A prepared for Image Forming Layer Forming Liquid Coating Composition 1 in an amount of 0.974 g, 2.989 g, 13.543 g, 3.570 g, 6.461 g, and 0.576, respectively, whereby Image Forming Layer Forming Liquid Coating Composition 3 was prepared.

(Coating the Image Forming Layer Side)

Image Forming Layer Forming Liquid Coating Composition 3 prepared employing the aforesaid method and the protective layer forming liquid coating composition which was the same as Image Forming Material 1 were subjected to multilayer coating onto a plasma treated surface of the support provided with the aforesaid backing layer which was the same as Image Forming Material 1, while employing an extrusion coater, and subsequently dried by 75° C. air flow, whereby Image Forming Material 3 was prepared. The thickness of the image forming layer was controlled to result in a silver amount of 1.45±0.03 g/m², and the protective layer was controlled to an amount of 2.00±0.05 g/m².

### IMAGE FORMING MATERIAL 4

### Image Forming Material Comprised of a White Reflective Support

While stirring, added to 83.0 g of methyl ethyl ketone were 8.42 g of cellulose acetate propionate (CAP482-20, manufactured by Eastman Chemical Co.) and 0.45 g of polyester resin (Vylon 280, manufactured by TOYOBO Co., Ltd.) and were dissolved. While stirring, added to the resulting solution was a fluorine based surfactant solution which was separately prepared by dissolving in 4.32 g of methanol, 0.64 g of a fluorine based surfactant (Surfron S-381 (effective component of 70 percent), manufactured by Asahi Glass Co, Ltd.) and 0.23 g of a fluorine based surfactant (Megafag F120K, manufactured by Dainippon Ink and Chemicals, Inc.). Finally, while stirring, successively added were 7.5 g of silica (Sylophobic 4004, manufactured by Fuji Silysia Chemical Ltd.), which was dispersed in methyl ethyl ketone at a concentration of one percent employing a dissolver type homogenizer) and 1.78 g of an isocyanate compound (Coronate C-3041, manufactured by Nippon Polyurethane Industry Co., Ltd.) which was diluted with methyl ethyl ketone to result in a solid concentration of 20 percent by weight, whereby a backing layer forming liquid coating composition was prepared.

<Coating of Backing Layer>

Subsequently, employed as a support was a 188 μm thick polyester based synthetic paper (Crisper G1212, manufactured by TOYOBO Co., Ltd). One surface was then subjected to a plasma treatment under a high-frequency output of 4.5 kW, a frequency of 5 kHz, a treatment time of 5 seconds and gas conditions at a volume ratio of argon, nitrogen, and hydrogen of 90, 5, and 5 percent respectively, employing a batch type atmospheric plasma treatment apparatus (AP-I-H-340, manufactured by E. C. Kagaku Co., Ltd.). Subsequently, the opposite surface was subjected to a corona discharge treatment (at 40 W/m²·minute) and the aforesaid backing layer forming liquid coating composition was applied onto the corona discharged surface to result in a dried layer thickness of 3.50 μm, employing an extrusion coater and subsequently dried, whereby a backing layer was formed.

<Preparation of Image Forming Layer Forming Liquid Coating Composition 4>

In Image Forming Layer Forming Liquid Coating Composition 1, a solution, to which Infrared Dye 1 used in Additive Solution 3 was not added, was prepared and the resulting solution was designated as Additive Solution 8. Subsequently, while stirring, Additive Solutions 1, 2, 8, 4, and 5 were successively added to Solution A prepared for Image Forming Layer Forming Liquid Coating Composition 1 in an amount of 0.974 g, 2.989 g, 13.543 g, 3.570 g, and 6.461 g, respectively, whereby Image Forming Layer Forming Liquid Coating Composition 4 was prepared.

(Coating the Image Forming Layer Side)

Image Forming Layer Forming Liquid Coating Composition 4, prepared employing the aforesaid method and the protective layer forming liquid coating composition which was the same as Image Forming Material 1, were subjected to multilayer coating onto a plasma treated surface of the support provided with the aforesaid backing layer which was the same as Image Forming Material 1, while employing an extrusion coater, and subsequently dried by 75° C. air flow, whereby Image Forming Material 4 was prepared. The thickness of the image forming layer was controlled to result in a silver amount of 1.45±0.03 g/m², and the protective layer was controlled to an amount of 2.00±0.05 g/m².

### IMAGE FORMING MATERIAL 5

### Image Forming Material which is Comprised of a Non-Tinted Transparent Support

Image Forming Material 5 was prepared in the same manner as Image Forming Material 1, except that the support employed in Image Forming Material 1 was replaced with a 188 μm thick biaxially oriented polyethylene terephthalate film of a visual transmission density with 0.006 (measured by PDA-65, manufactured by Konica Corp. to three places of decimals). Incidentally, the thickness of the image forming layer was controlled to result in a silver amount of 1.85±0.05 g/m², and the protective layer was controlled to an amount of 2.00±0.05 g/m².

Image Forming Materials 1-5, prepared as above, were exposed and thermally developed based on a calibration curve, employing a dry imager (Konica Dry Imager DryPro722, manufactured by Konica Corp.). The resulting maximum densities are shown in Table 1. With regard to densities, either transmission density or reflection density was determined, employing a densitometer (PDA-65, manufactured by Konica Corp.), depending on the form which is suitable for viewing image forming materials.

TABLE 1

| Image | | |
|---|---|---|
| Forming | Maximum | |
| Material | Density | Remarks |
| 1 | 3.32 | Image Forming Material comprised of a |
| | | blue tinted transparent support |
| 2 | 3.32 | Image Forming Material which results |
| | | in a different color tone from Image |
| | | Forming Material 1 |
| 3 | 3.98 | Image Forming Material which results |
| | | in a different maximum density from |
| | | Image Forming Material 1 |
| 4 | 2.39 | Image Forming Material comprised of a |
| | | white reflective support |
| 5 | 3.18 | Image Forming Material comprised of a |
| | | non-tinted transparent support |

The spectral absorption of the colored area of Image Forming Materials 1 and 2 was determined employing a spectrophotometer (U-3300, manufactured by Hitachi, Ltd.) When Imaage Forming Material 2 was compared to Image Forming Material 1, the coloration peak of the color tone controlling agent was noticed. Therefore, they were clearly different image forming materials based on the appearance of color tone.

The image forming apparatus, the image forming method and the image forming system of the present invention will now be described with reference to the embodiments.

### PREFERRED EMBODIMENTS OF THE INVENTION

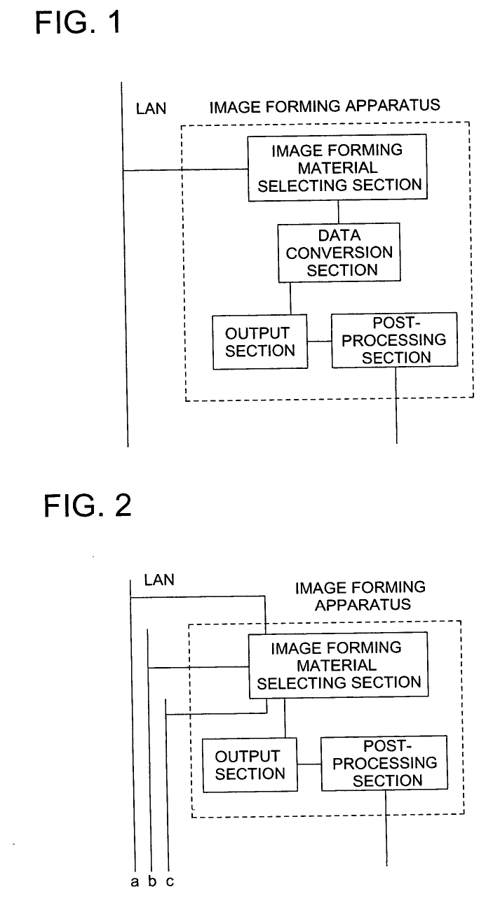

FIGS. 1-3 each shows a flow chart of the process of the image forming apparatus of the present invention.

In FIG. 1, digital data, as medical image data, are transmitted to an image forming apparatus connected to a network, and by employing an image forming material selecting means in the image forming apparatus, one image forming material is selected from at least two image forming materials, which have different characteristic (such as color tone or maximum density of the image), in the image forming apparatus. In such a case, the image forming material may be selected based on the transmitted digital data. Alternatively, selection may be made in the image forming apparatus based on image forming material selecting information which is attached to digital data as supplemental information. Subsequently, in order to convert digital data to be suitable for the selected image forming material, the original digital data are subjected to data conversion, employing an image data-converting section and the converted data (outputting image data) are then outputted from an outputting section. Subsequently, the final image is formed by post-processing the finally selected image forming material, employing a post-processing section. The data conversion, as described herein, refers, for example, conversion of the gradient of the original image data, conversion of the number of gradation, conversion of color, and conversion of LUT (Look-Up Table). The outputting section, as described herein, refers to a hard copy outputting section which records medical image data onto image forming materials. In other drawings explained hereafter, each the data conversion and the outputting section refers the same meaning as in FIG. 1.

In FIG. 2, in the same manner as FIG. 1, digital data, as medical image data, are transmitted to an image forming apparatus connected to a plurality of networks, and by employing an image forming material selecting means in the image forming apparatus, one image forming material is selected from at least two image forming materials, which each have a different characteristic, in the image forming apparatus. In such a case, in the same manner as the case of FIG. 1, the image forming material may be selected based on the transmitted digital data. Alternatively, selection may be made in the image forming apparatus based on image forming material selecting information which is attached to digital data as supplemental information. Subsequently, digital data are outputted to the selected image forming material from an outputting section and the final image is formed by post-processing the finally selected image forming material, employing a post-processing section.

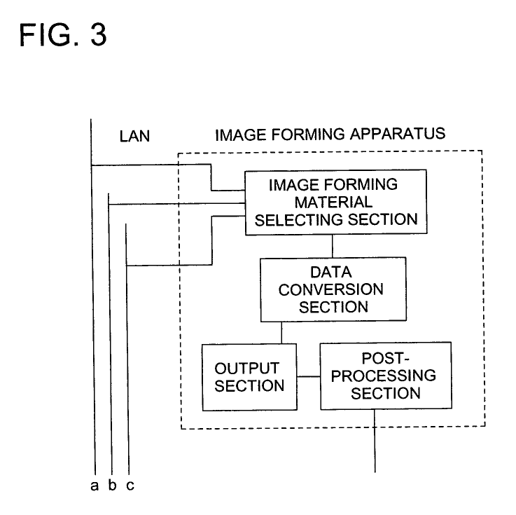

FIG. 2 is described in a form in which the image forming apparatus is connected to a plurality of networks represented by a, b, and c. However, a case in which even though there is a plurality of medical image database-managing apparatuses (also referred to as medical image database servers), which manage information, one network cable is employed, is in the scope of the present invention. This also applies to FIG. 3 described below.

In FIG. 3, in the same manner as in FIG. 1, digital data, as medical image data, are transmitted to an image forming apparatus connected to a plurality of networks, and by employing an image forming material selecting section in the image forming apparatus, one image forming material is selected from at least two image forming materials, which each have a different characteristic, in the image forming apparatus. In such a case, in the same manner as in the case in FIG. 1, an image forming material may be selected based on the transmitted digital data. Alternatively, selection may be made in the image forming apparatus based on image forming material selecting information which is attached to digital data as supplemental information. Subsequently, in order to convert digital data to be suitable for the selected image forming material, the original digital data are subjected to data conversion, employing a data converting section and the converted data are outputted from an outputting section. Subsequently, the final image is formed by post-processing the finally selected image forming material, employing a post-processing section.

As noted above, in image forming apparatuses and image forming method of the present invention shown in FIGS. 1-3, there are at least two image forming materials, which each have different characteristic, in the image forming apparatus. As a result, when images are to be outputted which require different characteristic, it is possible to output images without exchanging an image forming material to new one.

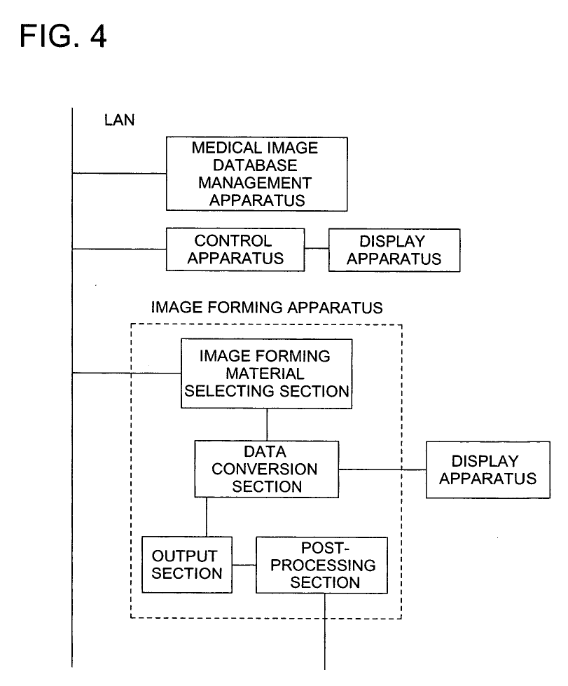

Further, FIG. 4 shows a case in which a medical image database managing apparatus, which manage information, and an image forming apparatus is connected with one network cable. Data to be further outputted are data which has been processed by a data control apparatus which is in the separate network cable, and a display is provided to confirm the medical image data which have been subjected to data conversion, employing a data converting section in the image forming apparatus.

Aforesaid FIGS. 1-4 are described as an image forming apparatus in which at least two image forming materials, which differ in color tone or in maximum density of images are placed for an example. Image forming materials may include ones which are used to prepare reflection images and the other which is used to prepare transparent images. In such cases, image forming material selecting section shown in FIGS. 1-4 make it possible to select, as required, transparent image forming materials or reflection image forming materials.

The image recording apparatus capable of outputting at least two image forming materials will now be specifically described based on drawings. However, when included in the scope of the present invention, the image recording apparatus is not limited to the one shown in the drawing.

In the image forming apparatus of the present invention, as an outputting section which outputs at least two image forming materials each having a different characteristic, it is possible to select and use any of the appropriate digital data outputting section, known in the art, such as a heating device employing a thermal head, and a light writing device employing a laser beam. Of these, from the viewpoint of simplicity and operation of the apparatus, the light writing means, employing a laser beam, is preferred, and an outputting section which outputs a laser beam directly onto an image forming material is more preferred.

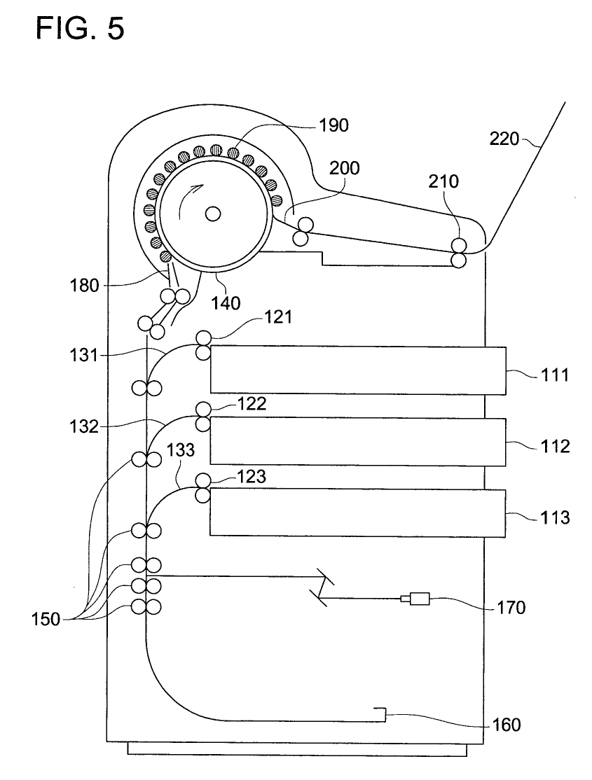

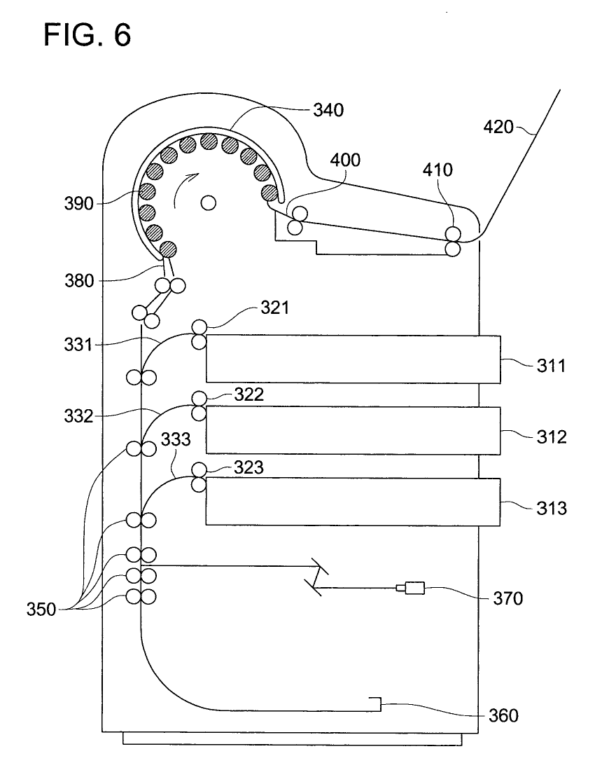

FIGS. 5 and 6 are schematic views of an image forming apparatus capable of forming images onto 3 types of image forming materials.

Further, FIG. 5 is a schematic view of the case in which a thermal treatment section is comprised of a heating roller or a heating-pressing roller in an image forming apparatus in which a post-processing section is a thermal treatment device, while FIG. 6 is a schematic view of a case in which the thermal treatment section is a heating block.

In FIG. 5, image forming material 1, comprised of a blue tinted transparent support, is housed in tray 111. When recording is initiated, feeding is carried out employing feeding roller 121 and a sheet is fed from tray 111. Then, the sheet is housed in temporary storage section 160 via conveyance rollers 150. Subsequently, the image recording martial is fed from temporary storage section 160, employing conveyance rollers 150 and during conveyance, is exposed to a laser beam emitted from laser beam source 170, based on digital data. The image forming material on which a latent image is formed is lead to heating roller 140 by image forming material guiding plate 180 and is thermally developed while interposed by facing rollers 190 and heating roller 140. Subsequently, developed image forming material is separated from heating roller 140 by separating plate 200, and is ejected to image stock section 220 on the exterior of the apparatus, employing ejection roller 210. Further, image forming material 2 which differs in color tone of images from image forming material 1 is housed in tray 112. When recording is initiated, feeding is carried out employing feeding roller 122 and a sheet is fed from 112. Then, the sheet is housed in temporary storage section 160 via conveyance rollers 150. Subsequently, the image recording martial is fed from temporary storage section 160, employing conveyance rollers 150 and during conveyance, is exposed to a laser beam emitted from laser beam source 170, based on digital data. The image forming material on which a latent image is written is lead to heating roller 140 by image forming material guiding plate 180 and is thermally developed while interposed by facing rollers 190 and heating roller 140. Subsequently, the developed image forming material is separated from heating roller 140 by separating plate 200, and is ejected to image stock section 220 on the exterior of the apparatus, employing ejection roller 210. Still further, image forming material 3 which differs in maximum density from image forming material 1 is housed in tray 113. When recording is initiated, feeding is carried out employing feeding roller 123 and a sheet is fed from tray 113. Then, the sheet is housed in temporary storage section 160 via conveyance rollers 150. Subsequently, the image recording martial is fed from temporary storage section 160, employing conveyance rollers 150 and during conveyance, is exposed to a laser beam emitted from laser beam source 170, based on digital data. The image forming material on which a latent image is formed is lead to heating roller 140 by image forming material guiding plate 180 and is thermally developed while interposed by facing rollers 190 and heating roller 140. Subsequently, developed image forming material is separated from heating roller 140 by separating plate 200, and is ejected to image stock section 220 on the exterior of the apparatus, employing ejection roller 210.

On the other hand, in FIG. 6, image forming material 5, comprised of a non-blue tinted transparent support, is housed in tray 311. When recording is initiated, feeding is carried out employing feeding roller 321 and a sheet is fed from tray 311. Then, the sheet is placed in 360 which temporarily stores the sheet via conveyance rollers 350. Subsequently, the image recording martial is fed from 360 in which the image forming material has been housed, employing conveyance rollers 350 and during conveyance, is exposed to a laser beam emitted from laser beam source 370, based on digital data. The image forming material on which a latent image is formed is lead between facing rollers 390 and heating roller 340 by guiding plate 380 and is thermally developed while interposed by facing rollers 390 and heating roller 340. Subsequently, developed image forming material is separated from heating roller 340 by separating plate 400, and is ejected to image stock section 420 on the exterior of the apparatus, employing ejection roller 410. Further, image forming material 2 comprised of a blue tinted transparent support is housed in tray 312. When recording is initiated, feeding is carried out employing feeding roller 322 and a sheet is fed from 312. Then, the sheet is placed in 360 which temporarily stores the sheet via conveyance rollers 350. Subsequently, the image recording martial is fed from 360 in which the image forming material has been housed, employing conveyance rollers 350 and during conveyance, is exposed to a laser beam emitted from laser beam source 370, based on digital data. The image forming material on which a latent image is formed is lead between facing rollers 390 and heating roller 340 by guiding plate 380 and is thermally developed while interposed by facing rollers 390 and heating roller 340. Subsequently, developed image forming material is separated from heating roller 340 by separating plate 400, and is ejected to image stock section 420 on the exterior of the apparatus, employing ejection roller 410. Still further, image forming material 4 which comprised of a white reflection support is housed in tray 313. When recording is initiated, feeding is carried out employing feeding roller 323 and a sheet is fed from 313. Then, the sheet is placed in 360 which temporarily stores the sheet via conveyance rollers 350. Subsequently, the image recording martial is fed from 360 in which the image forming material has been housed, employing conveyance rollers 350 and during conveyance, is exposed to a laser beam emitted from laser beam source 370, based on digital data. The image forming material on which a latent image is formed is lead between facing rollers 390 and heating roller 340 by guiding plate 380 and is thermally developed while interposed by facing rollers 390 and heating roller 340. Subsequently, developed image forming material is separated from heating roller 340 by separating plate 400, and is ejected to image stock section 420 on the exterior of the apparatus, employing ejection roller 410.

Selected and employed as lasers employed for scanning exposure based on digital data in the present image forming apparatus are solid lasers such as a ruby laser, a YAG laser, or a glass laser; gas lasers such as a He—Ne laser, an Ar ion laser, a Kr laser, a CO₂ laser, a Co laser, a He—Cd laser, an N₂ laser, or an excimer laser, semiconductor lasers such as an InGaP laser, an AlGaAS laser, a GaAsP laser, an InGaAs laser, an InAsP laser, a CdSnP₂ laser, or a GaSb laser, chemical lasers, and dye lasers, while matching to the use. While considering maintenance as well as the overall size of laser beam sources, of these, it is preferable to employ semiconductor lasers of an oscillation wavelength of 700-1,200 nm, and more preferably 750-850 nm, from the aspect of cost.

When an image forming material is subjected to laser scanning, the beam spot diameter on the exposed surface of the aforesaid material is customarily in the range of 4-75 μm in terms of the short axis diameter, and in the range of 4-100 μm in terms of the long axis diameter in the laser used in a laser imager. It is possible to set the laser beam scanning rate at an optimal value for each image forming material, depending on the inherent speed of the image forming material in the laser oscillation wavelength and the laser power.

Further, in the laser employed for the aforesaid scanning exposure, by employing the image forming material of the present invention, it is possible to produce clear images without interference fringes by adjusting the angle between the exposure surface and the laser beam, the wavelength of the laser beam, and the number of lasers. Incidentally, the aforesaid means may be employed individually or in combinations of at least two embodiments.

Still further, when a latent image is formed on the image forming material employing laser scanning, it is preferable to expose the image forming layer coated side.

As a first embodiment to exhibit the aforesaid effects, images are formed by scanning exposure, employing a laser beam so that the angle between the exposed surface, of the image forming material and the laser beam does not become substantially vertical. By deviating the incident angle from verticality as noted above, even though reflected light forms on the face between layers, differences in the optical path, which reaches the image forming layer, increase. As a result, interference fringes tend not to occur due to scattering and attenuation of a laser beam through the optical path. Incidentally, “does not become substantially vertical”, as described herein, means that during laser scanning, the angle which is nearest to verticality is not 90 degrees in both the primary scanning direction and the secondary scanning direction. The aforesaid angle is preferably 55-88 degrees in either the primary scanning direction or the secondary scanning direction, and is more preferably 60-86 degrees.

Further, in a second embodiment, images are formed by scanning exposure, employing a longitudinal multi-laser in which the wavelength of exposure light is not a single. When scanning is performed employing the aforesaid longitudinal multi-laser beam, the formation of interference fringes decreases compared to the scanning laser beam of a single longitudinal mode. Longitudinal multi-laser, as described herein, means that the wavelength is not a single one. The distribution of wavelengths of exposure light is preferably at least 5 nm, and is more preferably at least 10 nm. The upper limit of the distribution of the wavelengths of exposure light is not particularly limited, but is customarily about 60 nm.

Further, in a third embodiment, images are formed by scanning exposure while employing at least two lasers. An image forming method which utilizes a plurality of lasers is employed as an image writing means in laser printers as well as digital copiers in which a plurality of lines of an image is written in one scanning path to meet requirements to achieve a high production rate of image writing, which is disclosed, for example, in Japanese Patent Application Open to Public Inspection No. 60-166916. The aforesaid method is performed in such a manner that a laser beam emitted from a light source unit is deflected and scanned and focused onto a photoreceptor via an fθ lens and the like, and used in a laser scanning optical apparatus which utilizes the same principle as that in laser imagers.

Laser beam focusing onto a photoreceptor in an image writing means of laser printers, as well as digital copiers, is performed in such a manner that the following laser beam is focused while shifted by one line from the previous focused position, for use in which a plurality of lines of an image is written each time, employing one scanning. Specifically, two laser beams approach each other at an interval of an order of several 10 μm on the image surface in the secondary scanning direction. Printing density is 400 dpi (in the present invention, the number of dots per inch, or per 2.54 cm is designated as dpi) and pitches in the secondary scanning direction of 2 beams is 63.5 μm and 42.3 μm at 600 dpi. Being different from such a method in which shift equivalent to resolution is performed in the secondary scanning direction, in the present invention, it is characterized in that an image is formed by focusing at least two laser beams onto the same position while varying the incident angle. In such cases, it is preferable to satisfy the relationship of 0.9×E≦En×N≦1.1×E, wherein E represents the exposure light energy on the exposed surface when written by a single laser of wavelength λ (nm), N represents the number of lasers having the same wavelength λ, and En represents the same exposure energy. By satisfying the aforesaid relationship, sufficient energy on the exposed surface is assured. However, reflection of each laser beam on the image forming layer decreases due to the low exposure energy of the laser, whereby formation of interference fringes is minimized.

Incidentally, in the foregoing, a plurality of lasers having the same wavelength λ was employed, but those having different wavelengths may also be employed. In this case, it is preferable that the following relationship is sufficed with respect to λ [nm].

(λ−30)<λ1, λ2, . . . λn≦(λ+30)

In the aforesaid image forming apparatus, listed as thermal processing apparatuses employed in the case in which a post-processing section is a thermal processing device, are a heating roller and a heating-pressing roller as shown in FIG. 5, as well as a heating block shown in FIG. 6. The roller surface temperature of the roller-shaped thermal processing apparatus shown in FIG. 5 is customarily 115-135° C., and is preferably 120-130° C. Contact time is customarily 8-30 seconds, and is preferably 10-20 seconds, while linear pressure is customarily 0-50 N/cm, and is preferably 0-10 N/cm. Further, in the heating block-shaped thermal processing apparatus, the temperature near the backing layer side of the image forming material is customarily 115-140° C., and is preferably 120-130° C., while contact time is customarily 8-30 seconds, and is preferably 10-20 seconds.

In either thermal processing device, from the viewpoint of fogging and sharpness, it is preferable that the relationship of 1,200≦t×T≦2,600 (second·° C.) is held, wherein t (in seconds) represents the thermal processing time of an image forming material and T (in ° C.) is the surface temperature of the thermal processing means which the image forming material contacts. Further, it is more preferable that the relationship of 1,480≦t×T≦1,860 (second·° C.) is held.

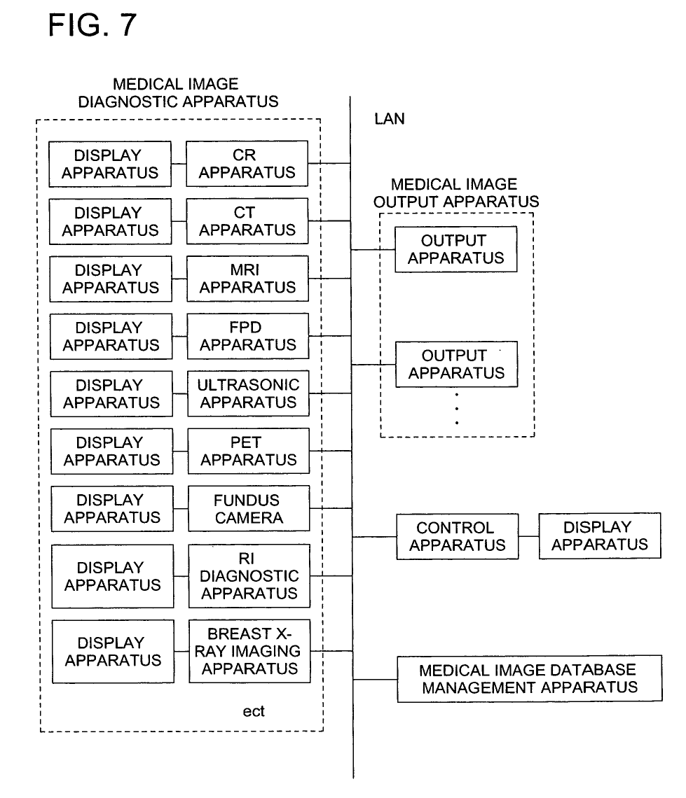

In a network related to medical image data in a medical organization, for example, as shown in FIG. 7, medical image diagnostic apparatuses such as a CR apparatus and an MRI apparatus are connected with an LAN cable. Image data which are obtained by these apparatuses are collected employing a medical image database management apparatus, and it is possible to recall any data employing a control apparatus. Further, the control apparatus is provided with a display apparatus. After confirming images employing the display apparatus, it is possible, if desired, to output the image data to an output apparatus. Further, in addition to the aforesaid medical image database, other medical management systems such as an electronic clinical card system and an accounting system are occasionally also connected.

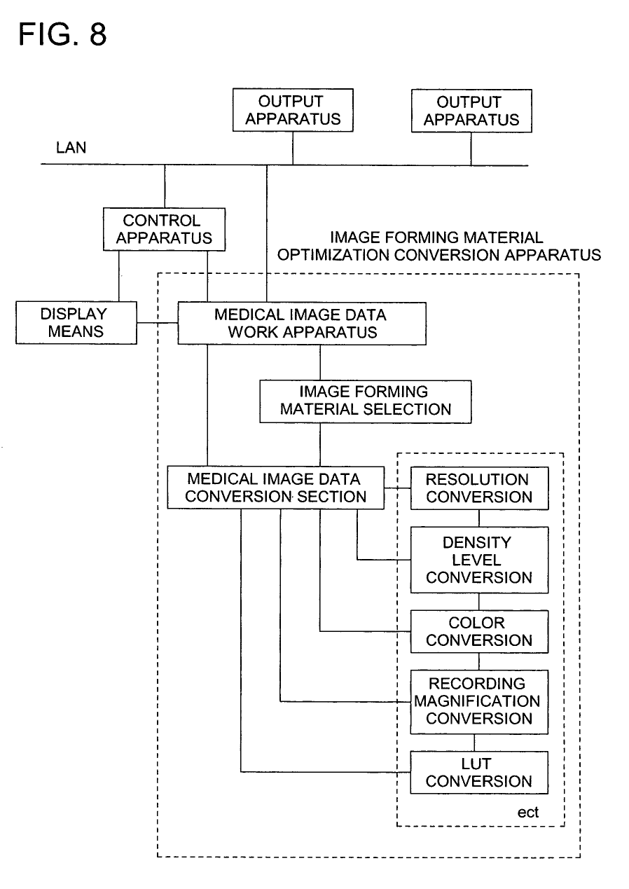

The image forming system of the present invention is characterized as follows. A section is included in which medical image data are converted to data which are suitable for one of at least two image forming materials having a different characteristic. The aforesaid conversion is primarily decided by selecting the image forming material. By transmitting medical image data which are converted to the suitable form for the aforesaid selected image forming material, images are then formed on the image forming material. Specifically, as shown in FIG. 8, various types of medical image data, which is sent from a non-illustrated inputting section and have been managed by the medical image database managing apparatus, are selected and adjusted, employing a control apparatus and the resulting medical image data are transmitted to an image forming material optimization converting apparatus, whereby one of at least two image forming materials being different from each other, is selected employing a medical image data processing apparatus. Subsequently, data are automatically converted by a medical image data converting section to a data which is suitable for the aforesaid selected image forming material, and if desired, after confirming the resulting processed work data, utilizing a display section, the work data are transmitted directly, or via a control apparatus, to an outputting apparatus which outputs images, and data are outputted from the aforesaid output apparatus.

When as image forming materials, there are an image forming material which is employed to form reflection images and another image forming material which is employed to form transmission images, at least one of these image forming materials is selected. Subsequently, data are automatically processed employing a data conversion means which is suitable for the selected image forming material. If desired, after confirming the aforesaid processed work data employing a display means, work data are transmitted directly, or via a control apparatus, to an output apparatus which outputs images, whereby data are outputted from the aforesaid output apparatus.

Suitably selected and employed as device, which are employed in the aforesaid medical image data converting section may utilize medical image processes known in the art, various conversion processes such as a printing image process which are employed for digital data processing, and further conversion processes described in Japanese Patent Application Open to Public Inspection Nos. 8-111816, 9-94243, 9-179977, 10-171979, 11-66280, 11-161770, 2000-11146, 2000-67136, 2000-67226, 2001-285627, 2002-10139, 2002-19197, 2002-144607, 2002-158863, 2002-158866, and 2002-171411. Of these, it is preferable that at least one of the conversion processes such as a gradient-converting process, a gradation number-converting process, a color converting process, or an LUT-converting process is included. Incidentally, the above embodiment is explained by using the medical image data, which is managed by the medical image data managing apparatus, however, the medical image data, which is sent from the inputting section, may be directly transferred to the image forming material optimization converting apparatus.

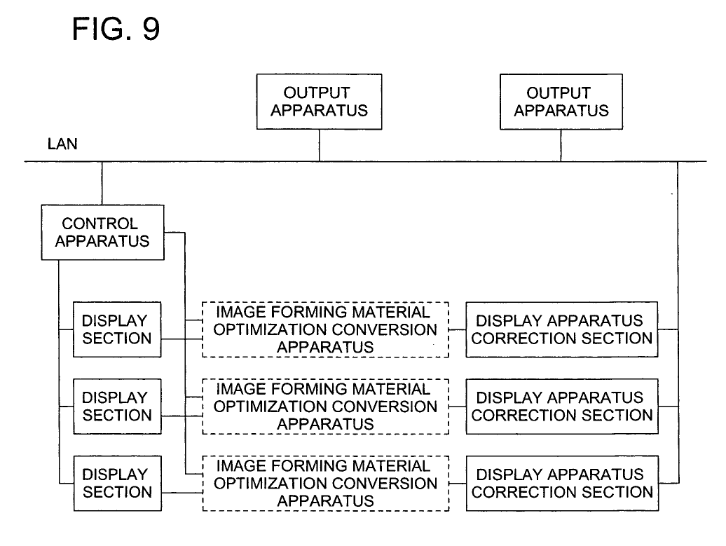

Further, in the case of tonal images such as medical images, gradation of images displayed by a display section occasionally differs from that outputted on the output apparatus. Therefore, in FIG. 9, the gradation of images displayed by a display section which displays the converted data in the image forming material optimization conversion apparatus from the control apparatus is matched to the gradation of images which are outputted from the output apparatus. In other words, it refers to an image forming method, in which a display apparatus correction section which corrects gradation, is further provided. In such cases, medical image work data may be transmitted to the output apparatus from the display apparatus correction section to output data from the output apparatus. Alternatively, data corrected by the display apparatus correction section may be outputted from the output apparatus via the control apparatus.

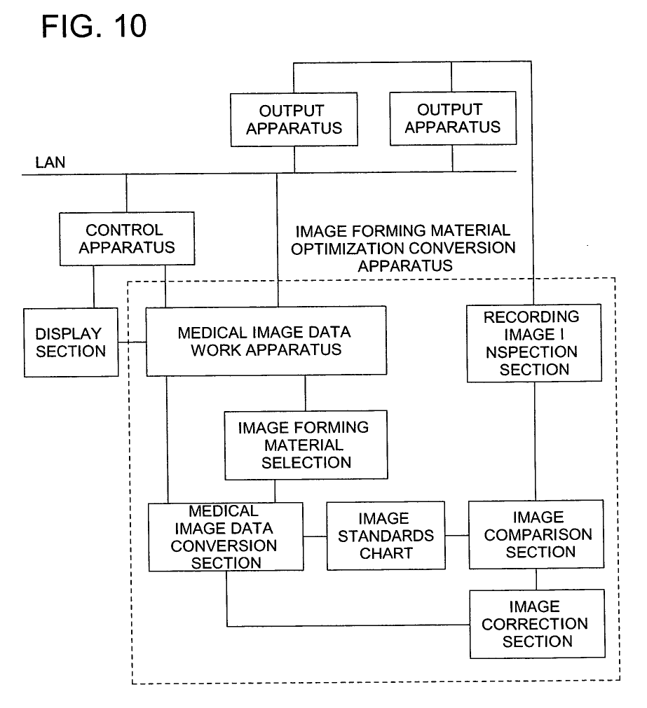

Still further, even though the aforesaid image forming material optimization converting apparatus is employed, occasionally desired output data are not provided due to the temperature in the interior of the output apparatus, differences in the environments in which the output apparatus is installed, or seasonal variations. In such cases, it is preferable that output data are inspected and after correcting the data based on the inspection results, the resulting data are again outputted by the output apparatus. Specifically, for example, as shown in FIG. 10, medical image data which are managed by the medical image database management apparatus are selected and adjusted employing the control apparatus. Subsequently, the resulting data are transmitted to the image forming material optimization converting apparatus, and by employing a medical image data work apparatus, selected is one of at least two image forming materials being different from each other (for example, the image forming materials each having different color tone or maximum density of the image when the image is formed in the same condition, or image forming materials, in which one has a transparent support and the other has a reflective support). Subsequently, data are automatically processed employing the medical image data converting section to a data, which is suitable for the selected image forming material, and if desired, the processed work data are confirmed employing the display section. Thereafter, the medical image work data are transmitted directly or via the control apparatus to the output apparatus and data are outputted from the output apparatus. The resulting outputted data are inspected employing a recording image checking section. By comparing image standard charts, which are provided in the medical image data converting section, corresponding to each image forming material to data which are obtained by the aforesaid recording image checking section, employing an image comparison section, and the resulting difference is corrected by an image correction section. Thereafter, the corrected integration data are transmitted directly or via the control apparatus to the output apparatus from the image forming material optimization converting apparatus, whereby data are outputted from the output apparatus. Incidentally, if desired, the aforesaid correction may be repeated a plurality of times.

As output apparatuses to achieve the aforesaid image recording method, it is preferable to employ any of the image forming apparatuses capable of selecting one of at least two image forming materials each having a different characteristic, and further, the image forming apparatus specifically explained as the image forming apparatuses of FIGS. 1 to 6 are preferably used. As the medical inputting apparatus used in the above-explained medical image data forming system, known digital inputting apparatus can be used, appropriately. Among them, above-described CR apparatus, CT apparatus, MRI apparatus, FPD apparatus, ultrasonic diagnosis apparatus, PET apparatus, fundus camera, and RI diagnosis apparatus are preferably employed. Further, as the image forming system of the present invention, it is preferable that plural inputting apparatus are connected to one network since unified process can be conducted to a medical image data.

### Effects of the Invention

According to the present invention, it is possible to provide an image forming apparatus, an image forming method, and an image forming system, which are suitable for the simultaneous use of at least two image forming materials which are different in at least one characteristic.

What is claimed is:

1. An image forming apparatus for forming an image based on digitalized medical image data, comprising:
a first image forming material-supplying section supplying a first image forming material;
a second image forming material-supplying section supplying a second image forming material, which is different from the first image forming material;
a selecting section selecting an image forming material to be output from the first and second image forming materials;
a converting section converting the digitalized medical image data to an outputting image data, which is suited to the selected image forming material;
an outputting section outputting the outputting image data onto the selected image forming material; and
a post-processing section conducting a post-processing to the selected image forming material to form a final image.

2. The image forming apparatus of claim 1, wherein each of the first image forming material and the second image forming material is a tray.

3. The image forming apparatus of claim 1, wherein the first image forming material and the second image forming material are different in a color tone or a maximum density from each other when an image is formed on each of the first image forming materials and the second image forming material in same condition.

4. The image forming apparatus of claim 1, wherein the first image forming material and the second image forming material are different in at least one of sensitivity, transmittance and a gradient from each other.

5. The image forming apparatus of claim 1, wherein one of the first image forming material and the second image forming material has a reflective support and the other has a transparent support.

6. The image forming apparatus of claim 1, wherein the outputting section is a photo-writing device utilizing a laser scan exposure.

7. The image forming apparatus of claim 6, wherein a system of the laser scan exposure of the photo-writing device is a laser scan exposing system in which an angle of an exposed surface and a laser beam is not substantially perpendicular, a longitudinally multi laser scan exposing system utilizing a laser beam has plural exposing wavelengths, or a laser scan exposing system in which the laser scan exposure is conducted by two or more of laser beams.

8. The image forming apparatus of claim 6, wherein an emission wavelength of a laser source utilized in the photo-writing device is in a range of 600 to 1200 nm.

9. The image forming apparatus of claim 8, wherein the emission wavelength is in a range of 750 to 850 nm.

10. The image forming apparatus of claim 1, wherein the post-processing section is a heat-processing device.

11. The image forming apparatus of claim 10, wherein the image forming apparatus suffices the following relationship,
1200≦t×T≦2600 [sec·° C.]
wherein t is a time period, the selected image forming material being subjected to a heat-processing; and T is a temperature of a surface of the heat-processing device where the selected image forming material contacts.

12. The image forming apparatus of claim 11, wherein the image forming apparatus suffices the following relationship,
1480≦t×T≦1860 [sec·° C.],
wherein t and T are the same as in claim 11.

13. The image forming apparatus of claim 1, wherein the converting section has at least one of a resolution-converting function, a gradient-converting function, a color-converting function and an LUT converting function.

14. The image forming apparatus of claim 13, wherein the converting section has at least one of the resolution converting function, the gradient-converting function and the color-converting function.

15. The image forming apparatus of claim 13, wherein the converting section has the LUT-converting function.

16. An image forming method for forming an image based on a digitalized medical image data, comprising:
selecting an image forming material to be output from plural image forming materials, which are different from each other;
converting the digitalized medical image data to an outputting image data, which is suited to the selected image forming material;
outputting the outputting image data onto the selected image forming material; and
conducting a post-processing to the selected image forming material after the outputting step to form a final image.

17. The image forming method of claim 16, wherein the plural image forming materials are different in a color tone or a maximum density from each other when an image is formed on each of the plural image forming materials in same condition.

18. The image forming method of claim 16, wherein the plural image forming materials are different in sensitivity, transmittance or gradient from each other.

19. The image forming method of claim 16, wherein the plural image forming materials include an image forming material having a reflective support and an image forming material having a transparent support.

20. The image forming method of claim 16, wherein the outputting step is conducted by a laser scan exposure.

21. The image forming method of claim 16, wherein the post-processing is a heat processing.

22. The image forming method of claim 21, wherein the image forming method suffices the following relationship,
1200≦t×T≦2600 [sec·° C.]
wherein t is a time period, the selected image forming material being subjected to a heat-processing; and T is a temperature of a surface of a heat-processing device where the selected image forming material contacts.

23. The image forming method of claim 22, wherein the image forming method suffices the following relationship,
1480≦t×T≦1860 [sec·° C.]
wherein t and T are the same as in claim 22.

24. The image forming method of claim 16, wherein the converting step is directly determined in accordance with a result of the selecting step.

25. The image forming method of claim 16, wherein the converting step includes at least one of the steps of converting resolution of the digitalized medical image data, converting gradient of the digitalized medical image data, converting color of the digitalized medical image data, and converting LUT of the digitalized medical image data.

26. The image forming method of claim 25, wherein the converting step includes at least one of the step of converting resolution of the digitalized medical image data, converting gradient of the digitalized medical image data and converting color of the digitalized medical image data.

27. The image forming method of claim 25, wherein the converting step includes the step of converting LUT of the digitalized medical image data.

28. The image forming method of claim 16, further comprising displaying the outputting image data on a displaying section.

29. The image forming method of claim 28, further comprising correcting the outputting image data for representing the outputting image data displayed by the displaying step onto the image forming material.

30. The image forming method of claim 16, further comprising checking the final image data whether a desired image has been obtained, correcting the outputting image data in accordance with a result of the checking step, outputting the corrected image data onto the image forming material, and conducting the post-processing to the image forming material.

31. The image forming method of claim 16, wherein the plural image forming materials each have a support having thereon a image forming layer, which contains a photosensitive silver halide, a photo-insensitive organic silver salt and a reducing agent, and a protective layer.

32. The image forming method of claim 31, wherein the plural image forming materials each have an intermediate layer, the image forming layer and the protective layer in that order on the support.

33. The image forming method of claim 31, wherein the plural image forming materials each have the image forming layer, a barrier layer and the protective layer in that order on the support.

34. An image forming system comprising:
a medical image data inputting apparatus including a medical image data-sending section;
a medical image data-managing apparatus including an image data-storing section and a medical image data-transferring section;
an image data-converting apparatus including an image forming material-selecting section, an image data-converting section and an outputting image data-transferring section; and
an outputting apparatus including a first image forming material-supplying section supplying a first image forming material, second image forming material-supplying section supplying a second image forming material being different from the first image forming material, an outputting section and a post-processing section,
wherein the medical image data-inputting apparatus, the medical image data-managing apparatus, the image data-converting apparatus and the outputting apparatus are connected via a network,
wherein the medical image data-inputting apparatus sends a digitalized medical image data by the medical image data-sending section to the medical image data-managing apparatus,
the medical image data-managing apparatus stores the medical image data in the image data-storing section; and transfers a medical image data to be output from the image data-storing section to the image data-converting apparatus,
the image data-converting apparatus selects an image forming material to be output from the first and second image forming materials by the selecting section; converts the transferred medical image data to an outputting image data being suited to the selected image forming material by the image data-converting section; and transfers the outputting image data with a result of the selection in the selecting section to the outputting apparatus by the outputting image-transferring section, and
the outputting apparatus outputs the outputting image data onto the selected image forming material, which is supplied from the first or second image forming material-supplying section in accordance with the result of selection, by the outputting section; and conducts a post-processing to the selected image forming material to form a final image.

35. The image forming system of claim 34, wherein the converting section has at least one of a resolution-converting converting function, a gradient-converting function, a color-converting function and an LUT-converting function.

36. The image forming system of claim 34, wherein the medical image data-inputting apparatus is a medical image diagnosis apparatus.

37. The image forming system of claim 34, wherein the image forming system comprises two or more of the medical image data-inputting apparatus connected via the network.
